(19) United States
(12) Patent Application Publication
(10) Pub. No.: US 2023/0321458 A1
(43) Pub. Date: Oct. 12, 2023

(54) THERAPEUTIC DEVICE UTILIZING ELECTROMAGNETIC RADIATION WITH OSCILLATING POLARIZATION STATE

(71) Applicant: The Board of Trustees of the Leland Stanford Junior University Office of the General Counsel, Stanford, CA (US)

(72) Inventors: John A. Fortkort, Austin, TX (US); Annelise E. Barron, Redwood Citty, CA (US)

(21) Appl. No.: 18/020,710

(22) PCT Filed: Aug. 12, 2021
(86) PCT No.: PCT/US2021/045829

§ 371 Date: Feb. 10, 2023

Related U.S. Application Data
(60) Provisional application No. 63/064,903, filed on Aug. 12, 2020.

(51) Int. Cl. A61N 5/06; G02B 27/28

(52) U.S. Cl. CPC A61N 5/0622; G02B 27/286; A61N 2005/0626; A61N 2005/0652; A61N 2005/0662

(57) ABSTRACT

In one aspect, a method is provided for performing light therapy on a subject. The method comprises providing a device which emits electromagnetic radiation that oscillates between at least first and second distinct polarization states; and illuminating the subject with the emitted electromagnetic radiation. In another aspect, a fixture is provided which comprises a first source of electromagnetic radiation which emits electromagnetic radiation in a first polarization state; a second source of electromagnetic radiation which emits electromagnetic radiation in a second polarization state which is distinct from said first polarization state; and an oscillator which oscillates electromagnetic radiation output by the fixture between at least said first and second polarization states.

### CROSS-REFERENCE TO RELATED APPLICATIONS

This application claims the benefit of priority of U.S. Patent Application No. 63/064,903, filed Aug. 12, 2020, having the same inventors and entitled “THERAPEUTIC DEVICE UTILIZING ELECTROMAGNETIC RADIATION WITH OSCILLATING POLARIZATION STATE,” which is incorporated herein by reference in its entirety.

### FIELD OF THE DISCLOSURE

The present application relates generally to light therapy, and more specifically to light therapy using a light source which emits light with an oscillating polarization.

### BACKGROUND OF THE DISCLOSURE

Neurons in the human body use action potentials (APs) to transmit information. These brief and uniform pulses of electrical activity are generated when the membrane potential of a neuron reaches a threshold value. The resulting pulses travel down the axon toward synapses and terminate at postsynaptic neurons, where they initiate postsynaptic currents (PSCs). The PSCs then summate to either trigger or inhibit new APs. The resulting sequence or “train” of APs may contain information based on various coding schemes and may produce various results. In simple motor functions such as muscle flexure, the strength at which the function occurs may depend solely on the firing rate of neurons. Other functions may rely on more complex temporal codes that are a function of the precise timing of single APs. These complex temporal codes may be tied to external stimuli (for example, those generated by the auditory system) or may be generated intrinsically by neural circuitry.

The human brain contains a large number of neurons. The electrochemical activity of neurons in generating the electrical currents required for APs occurs in a synchronized manner that is characterized by macroscopic oscillations. These oscillations may be characterized by their frequency, amplitude and phase, and may be monitored and depicted graphically in an electroencephalogram (EEG). The graphical depiction of these macroscopic oscillations in an EEG are often referred to as “brainwaves”.

Five common brainwave bandwidths (delta, theta, alpha, beta and gamma) have been identified in humans, each of which is associated with specific mental states. [Thompson, M., & Thompson, L. (2003). The neurofeedback book: An introduction to basic concepts in applied psychophysiology. Wheat Ridge, CO: The Association for Applied Psychophysiology and Biofeedback. Walter, V. J., & Walter, W. Grey]. Within these bandwidths, various sub-categories (such as, for example, high, low alpha and beta, and sensorimotor rhythm) have also been identified, which are associated with different mental activities.

By way of example, delta waves (0.5-3 Hz) are the dominant brainwaves observed during deep sleep. Theta waves (4-7 Hz) are typically associated with drowsy or relaxed states. Low alpha waves (8-10 Hz) are frequently associated with meditative states and inward thinking (such as, for example, daydreams and dissociation from external stimulation). High alpha waves (11-12 Hz) are associated with creativity and the alert but calm state needed for peak performance. Sensorimotor rhythms (13-15 Hz), which are frequently categorized as low beta, are believed to occur predominantly in the still state before a reactive psychomotor action. Low beta waves (16-20 Hz) are associated with intellectual activity and problem-solving. High beta waves (21-37 Hz) are found in emotional and anxious states. Gamma waves (38-42 Hz) are associated with attention and intense cognitive activity. [Id.]

An excess of brainwave activity in any of the foregoing bandwidths or sub-categories may also be associated with a particular state or condition. Thus, for example, excessive beta and gamma activity has been associated with hyper-aroused states, such as those occurring during stress, anxiety or insomnia. [Perlis, M. L., Merica, H., Smith, M. T. & Giles, D. E. (2001). Beta EEG activity and insomnia. Sleep Medicine Reviews, 5(5), 363-374].

Brainwave entrainment (sometimes referred to as brainwave synchronization or neural entrainment) may be utilized to modulate brainwaves to induce, for example, a particular mental state in a subject. Brainwave entrainment typically involves the manipulation of the frequency of brainwaves (or the associated patterns of firing of neural synapses) by suitable rhythmic or periodic external stimuli. Such stimuli may include auditory, visual, or tactile stimuli. The effectiveness of brainwave entrainment is believed to result from the tendency of the brain to naturally synchronize its brainwave frequencies with the oscillations of periodic external stimuli. Since (as noted above) particular patterns of neural firing have been associated with certain mental states, it is believed that brainwave entrainment may be utilized to induce desired states of consciousness by modulating brainwaves in a subject. Such states of consciousness may be those which are conducive, for example, to studying, sleeping, exercising, meditating, or doing creative work.

Early work in brainwave entrainment focused on the use of visual stimuli. However, Chatrian et al. found that brainwave entrainment could also be achieved with auditory stimuli alone (specifically, clicking sounds). [Chatrian, E. G., Peterson, M. C., & Lazarte, J. A. (1960). Responses to clicks from the human brain: Some depth electrograph observation. Electroencephalography and Clinical Neurophysiology, 12, 479-489]. This led to the discovery by Oster that binaural beats (which are produced by the simultaneous application of first and second distinct, single frequency sine wave tones to first and second ears of a subject, respectively) stimulate brain activity that corresponds to the rhythm of the difference in the two stimuli frequencies. [Oster, G. (1973). Auditory beats in the brain. Scientific American, 229, 94-102].

It has since been found that the foregoing modes of brainwave entrainment (namely, visual and auditory entrainment) may be combined. This technique, which is the subject of U.S. Pat. No. 3,838,417 (Charas), may be referred to variously as “audio visual stimulation” (AVS), “light and sound stimulation,” “audio photic stimulation,” or “audio visual entrainment.” AVS has been utilized in various clinical applications involving attention deficit disorder (ADD), academic performance, cognition, depression, stress management, tension, pain, PTSD, migraine headaches, hypertension, and stroke.

AVS brainwave entrainment may be open-loop or closed-loop. In closed-loop AVS brainwave entrainment, the subject is attached to EEG recording electrodes. Brain activity is measured through these electrodes and is used by the AVS device to provide light and sound stimulation based on the properties of the brain activity recorded. Hence, the stimulation is driven by the subject's brainwaves, and thus provides real-time feedback based on the activity of the user. This approach is termed “neurofeedback” and is typically conducted with the assistance of a clinician.

Open-loop AVS brainwave entrainment is not dependent on the subject's brainwave activity. In this approach, entrainment occurs in response to flickering light and audio tones of particular frequencies. Unlike the closed-loop approach, this form of AVS entrains brain activity in response to the designated frequencies (which are typically selected to induce a desired mental state), without any brain activity feedback provided to the AVS device. Various consumer products have been developed to implement open-looped AVS brainwave entrainment. These include, for example, the brainwave entrainment devices sold under the trademark EquiSync®.

Other types of light therapy have also been developed in the art that do not necessarily involve brainwave entrainment. For example, photobiomodulation therapy (PBMT) is a type of light therapy that utilizes non-ionizing electromagnetic energy to trigger photochemical changes in cellular structures that are receptive to photons. Various devices have been developed in the art to implement PBMT or processes related thereto. Examples of such devices are described, for example, in U.S. 2019/0246463A1 (Williams et al.)., U.S. US2019/0175936 (Gretz et al.), WO2019/053625 (Lim), U.S. U.S. 2014/0243933 (Ginggen), U.S. 2019/0142636 (Tedford et al.), U.S. Pat. No. 7,354,432 (Eells et al.), U.S. 2008/0091249 (Wang), U.S. Pat. No. 10,391,330 (Bourke et al.) and U.S. 2016/0129278 (Mayer). Various salutary effects have been ascribed to PBMT including, for example, promotion of tissue healing or regeneration, reduction in inflammation, and general analgesic effects.

### BRIEF DESCRIPTION OF THE DRAWINGS

FIG. 1 is a series of illustrations depicting right-handed (or clockwise) circularly polarized light (FIG. 1A) and left-handed (or counterclockwise) circularly polarized light (FIG. 1B) displayed without their vector components.

FIG. 2 is a series of illustrations depicting right-handed (or clockwise) circularly polarized light (FIG. 2A) and left-handed (or counterclockwise) circularly polarized light (FIG. 2B) displayed with their vector components.

FIG. 3 is a series of illustrations depicting the concepts of linear (FIG. 3A), circular (right-handed) (FIG. 3B) and elliptical (right-handed) (FIG. 3C) polarization.

FIG. 4 is a series of illustrations comparing linearly, circularly and elliptically polarized electromagnetic radiation in a first polarization state (FIG. 4A) to linearly, circularly and elliptically polarized radiation in a second polarization state (FIG. 4B). In FIG. 4A, the linearly, circularly and elliptically polarized electromagnetic radiation has an electric field confined to a first plane, while in FIG. 4B, the linearly, circularly and elliptically polarized electromagnetic radiation has an electric field confined to a second plane which is distinct from the first plane.

FIG. 5 is an illustration of the concepts of polarization by reflection and polarization by transmission.

FIG. 6 is an illustration of a device which utilizes a linear polarizer to transform unpolarized light into linearly polarized light, and which further utilizes a quarter-wave plate to transform the linearly polarized light into circularly polarized light having a left-handed orientation.

FIG. 7 depicts a polarization ellipse, the shape and orientation of which may be utilized to describe any fixed polarization of electromagnetic radiation. The shape and orientation of the polarization ellipse may be defined, respectively, by the axial ratio AR (that is, the ratio of major and minor axes of the ellipse) and the tilt angle t.

FIG. 8 depicts an LED package which may be utilized in the devices and methodologies disclosed herein.

FIG. 9 depicts an RGB LED which may be utilized in the devices and methodologies disclosed herein.

FIG. 10 depicts a first embodiment of a source of electromagnetic radiation which may be utilized in the devices and methodologies disclosed herein.

FIG. 11 depicts a second embodiment of a source of electromagnetic radiation which may be utilized in the devices and methodologies disclosed herein.

FIGS. 12-15 depict a light therapy unit which may be utilized to implement the devices and methodologies disclosed herein.

FIG. 16 is a graphical depiction of brainwaves from different frequency ranges. FIG. 1(6a) depicts brainwaves from the delta band. FIG. 16(b) depicts brainwaves from the theta band. FIG. 16(c) depicts brainwaves from the alpha band. FIG. 16(d) depicts brainwaves from the mu-rhythm band. FIG. 16(e) depicts brainwaves from the beta band. FIG. 16(f) depicts brainwaves from the gamma band.

FIG. 17 is an illustration of a liquid crystal (LC) device which may be utilized to create a light source which oscillates between two or more polarization states.

### SUMMARY OF THE DISCLOSURE

In one aspect, a method is provided for performing light therapy on a subject. The method comprises providing a device which emits electromagnetic radiation that oscillates between at least first and second distinct polarization states; and illuminating the subject with the emitted electromagnetic radiation.

In another aspect, a fixture is provided which comprises a first source of electromagnetic radiation which emits electromagnetic radiation in a first polarization state; a second source of electromagnetic radiation which emits electromagnetic radiation in a second polarization state which is distinct from said first polarization state; and an oscillator which oscillates electromagnetic radiation output by the fixture between at least said first and second polarization states.

In a further aspect, an LED array is provided which comprises a first set of LEDs which emit electromagnetic radiation in a first polarization state; a second set of LEDs which emit electromagnetic radiation in a second polarization state which is distinct from the first polarization state; and an oscillator which oscillates electromagnetic radiation output by the LED array between at least said first and second polarization states.

In yet another aspect, a method is provided for performing electromagnetic radiation therapy on a subject. The method comprises providing an electromagnetic radiation fixture equipped with an LED array containing (a) a first set of LEDs which emit electromagnetic radiation in a first polarization state, and (b) a second set of LEDs which emit electromagnetic radiation in a second polarization state; positioning the electromagnetic radiation fixture such that electromagnetic radiation emitted by the fixture is directed at the subject; and oscillating the LED array between first and second illumination states selected from the group consisting of
(a) a first illumination state in which the first set of LEDs are illuminated and the second set of LEDs are not illuminated, and a second illumination state in which the first set of LEDs are not illuminated and the second set of LEDs are illuminated,
(b) a first illumination state in which the first set of LEDs are powered on and the second set of LEDs are powered off, and a second illumination state in which the first set of LEDs are powered off and the second set of LEDs are powered on, and
(c) a first illumination state in which the power supply to the first set of LEDs is at a maximum and the power supplied to the second set of LEDs is at a minimum, and a second illumination state in which the power supply to the first set of LEDs is at a minimum and the power supply to the second set of LEDs is at a maximum, and
(d) a first illumination state in which the current supplied to the first set of LEDs is I₁₁ and the power supplied to the second set of LEDs is I₁₂, and a second illumination state in which the power supply to the first set of LEDs is I₂₁ and the power supply to the second set of LEDs is I₂₂, wherein I₁₁>I₂₁ and I₁₂<I₂₂.

### DETAILED DESCRIPTION

While several open-loop brainwave entrainment devices and methodologies have been developed to date, further improvement is needed in these devices. For example, typical existing open-loop AVS brainwave entrainment devices utilize an entrainment signal at a single frequency which, in some cases, may be varied over time. This approach may be utilized, for example, to gradually bring a subject into a state of restfulness characterized by inducing greater theta wave activity in the brain. In a typical implementation, this may be accomplished, for example, by beginning the entrainment process using a higher frequency signal, and then gradually lowering the frequency of the signal to within the theta range.

However, as previously noted, the human brain utilizes brainwaves whose frequencies fall within at least five common bandwidths (delta, theta, alpha, beta and gamma), each of which is associated with specific mental states. Hence, using single frequency entrainment may limit the technique to addressing only one of these bandwidths at a time.

Moreover, brainwaves commonly occur in more than one of these frequency bandwidths concurrently. For example, the hippocampus supports not only long term memory encoding and storage, but also plays a role in working memory maintenance of multiple items. While the neural mechanism underlying multi-item maintenance is not fully understood, theoretical work suggests that multiple items are maintained by neural assemblies synchronized in the gamma frequency range (25-100 Hz) that are locked to oscillatory activity (and in particular, to consecutive phase ranges of the oscillatory activity) in the theta frequency range (4-8 Hz). Indeed, cross-frequency coupling of the amplitude of high-frequency activity to the phase of slower oscillations has been found in both animals and in humans. Recent research suggests that simultaneous maintenance of multiple items in working memory is accompanied by cross-frequency coupling of oscillatory activity in the hippocampus, which is recruited during multi-item working memory. Moreover, maintenance of an increasing number of items is found to be associated with modulation of beta/gamma frequencies and amplitudes onto the theta band brain activity in both the frequency and amplitude of this lower frequency. This is consistent with the hypothesis that longer cycles are required for an increased number of representations by gamma cycles. Research also suggests that the precision of cross-frequency coupling predicts individual working memory performance. The foregoing supports the hypothesis that working memory in humans depends on a neural code using phase information. [See Axmacher N, Henseler M M, Jensen O, Weinreich I, Elger C E, Fell J. Cross-frequency coupling supports multi-item working memory in the human hippocampus. Proc Natl Acad Sci USA. 2010; 107(7):3228-3233].

Other work in the field supports the thesis that various functions of the brain are dependent on cross-frequency coupling of brainwaves from different frequency domains. For example, robust coupling has been observed between the high- and low-frequency bands of ongoing electrical activity in the human brain. In particular, the phase of the low-frequency theta (4 to 8 hertz) rhythm modulates power in the high gamma (80 to 150 hertz) band of the electrocorticogram, with stronger modulation occurring at higher theta amplitudes. Furthermore, different behavioral tasks evoke distinct patterns of theta/high gamma coupling across the cortex. The results indicate that transient coupling between low-frequency and high-frequency brain rhythms coordinates activity in distributed cortical areas, providing a mechanism for effective communication during cognitive processing in humans. [Canolty R T, Edwards E, Dalal S S, et al. High gamma power is phase-locked to theta oscillations in human neocortex. Science. 2006; 313(5793):1626-1628].

Other work has elicited the nature of specific types of cross-frequency coupling. For example, a considerable amount of work has focused on phase-amplitude coupling (PAC), a form of cross-frequency coupling where the amplitude of a high frequency signal is modulated by the phase of low frequency oscillations. [Munia, T. T. K., Aviyente, S. Time-Frequency Based Phase-Amplitude Coupling Measure For Neuronal Oscillations. Sci Rep 9, 12441 (2019)]. It has been suggested that PAC is responsible for integration across populations of neurons, with lower frequency brain activity controlling the information exchange between brain regions by modulating the amplitude of higher frequency oscillations. In particular, spatially distributed coherent oscillations are thought to provide temporal windows of excitability that allow for interactions between distinct neuronal groups. It has been hypothesized that this mechanism for neuronal communication is realized by bursts of high-frequency oscillations that are phase-coupled to a low frequency spatially distributed coupling oscillation. This mechanism requires multiple physiologically different interacting sources (one generating the low-frequency coupling oscillation and another generating phase-coupled high-frequency oscillations).

Support for the foregoing theory has been obtained using human intracranial EEG (iEEG) data, which provides evidence for multiple oscillatory patterns, as characterized on the basis of their spatial maps (topographies) and their frequency spectra. Indeed, the spatial maps for the coupling oscillations are found to be much more widespread than the ones for the associated phase-coupled bursts. Moreover, in the majority of the patterns of phase-amplitude coupling (PAC), phase-coupled bursts of high-frequency activity are synchronized across brain areas. In addition, working memory operations have been observed to affect the PAC strength in a heterogeneous way. In particular, working memory operations are found to increase the strength of some PAC patterns, while in others, working memory decreases it in the form of cross-frequency coupling where the amplitude of a high frequency signal is modulated by the phase of low frequency oscillations. [Maris, E., van Vugt, M., & Kahana, M. (2011). Spatially distributed patterns of oscillatory coupling between high-frequency amplitudes and low-frequency phases in human iEEG. Neuroimage, 54(2), 836-850].

In light of the foregoing, it will be appreciated that, while oscillatory brain activity reflects different internal brain states that may be characterized by the excitatory state of neurons and the synchrony among neurons, characterizing these states is complicated by the fact that different oscillations are often coupled (such as, for example, gamma oscillations nested in theta in the hippocampus). Moreover, changes in such coupling may reflect distinct mental states which may be characterized by oscillatory cycles based on distinct frequency and phase coupling. Consequently, single frequency brainwave entrainment may be insufficient or suboptimal in addressing these states, and its use may ignore potential advantages that may be attendant to entrainment in multiple regions simultaneously.

PCT/US21/42675 (Fortkort et al.), entitled “SYSTEMS AND METHODOLOGIES FOR TREATING OR PREVENTING PSYCHIATRIC DISORDERS WITH BRAIN ENTRAINMENT USING NESTED FREQUENCIES”, which was filed on Jul. 22, 2021, and which is incorporated herein by reference in its entirety, addresses the foregoing problem through the use in brainwave entrainment (and preferably, in open-loop AVS brainwave entrainment) of nested wave functions. In some applications, this may allow brainwave entrainment to simultaneously address distinct frequency regimes or distinct regions of the brain in a concerted fashion not unlike the native action of some neuronal processes.

Meanwhile, other work suggests that the polarization of light may be significant in the stimulation of biological processes. Thus, for example, DE3220218A1 (Fenyõ et al.) discloses a method and a device for the stimulation of biological processes associated with cell activity. The device is said to facilitate the healing of injuries of the body surface, such as wounds, ulcers and epithelial damage. In accordance with the methodology disclosed therein, the cell structure of a subject is irradiated with linearly polarized light of a pre-determined intensity having incoherent wavelength components above 300 nm. The device comprises a light source having a lamp which emits incoherent visible and/or infrared light, a deflection system which projects the light rays into a given treatment direction, a polarizer which is arranged in the beam path of the light deflected into the treatment direction, and (preferably) ultraviolet and infrared filters.

Subsequent light therapy devices have been developed in the art which sought to further build upon the findings of Fenyoe et al. These include the devices described in U.S. Pat. No. 5,001,608 (Kehrli et al.), U.S. Pat. No. 5,010,452 (Krebser et al.), HU222,162 (Fenyõ), WO1996004958 (Bolleter), WO1996004959 (Bolleter), and WO2011033329 (Fenyõ et al.).

This general technology has been commercialized by Sensolite Medical. According to the company's website [sensolite.com], the early work of Fenyõ et al. (see DE3220218A1 described above) revolved around the discovery of the stimulative effect polarized light has on all living biological systems, including a significant invigoration of the self-healing abilities of the human body when used in human therapy. According to the website, this result arises from the effect of polarized light on the regeneration, revitalization and harmonization of cell function. In particular, when the surface of the body is treated with polarized light, the light penetrates to a depth of 1 cm, thus reaching the blood stream via the capillary veins. This is said to result in a rapid transmission of the biological effect of the light stimulation throughout the entire body. In particular, the effect of the polarized light stimulation is said to travel through the circulatory system to all cells and vital organs of the body, including the heart, liver, stomach, spleen, kidneys and endocrine glands, thereby exerting a systemic effect on their function.

The foregoing website notes that polarized light significantly enhances the activity of the immune-competent cells, stabilizes the cell membrane of red blood cells, and enhances their ability to bind and retain oxygen. It further notes that treatment using polarized light significantly stimulates the activity of T-lymphocytes responsible for recognizing and defeating millions of faulty cells produced minute by minute in the human body that subsequently become responsible for serious illnesses and malignant deformations. Hence, polarized light therapy is said to prevent the development of serious illnesses and to facilitate and accelerate the recovery from protracted illnesses. It concludes that, through the enhanced ability of the red blood cells to bind and retain oxygen, more vital oxygen becomes available for each cell, organ and system thereby enhancing the efficiency of musculature and vital organs in growth and function. Hence, the use of polarized light (especially in a “body surround” implementation) is said to significantly strengthen the immunization of the entire body.

Some support for the therapeutic effects of polarized light may be found in the technical literature. Thus, for example, Tada et al. investigated the effect of polarized light on wound healing. They found that right circularly polarized light and linearly polarized light promoted the process of wound healing by increasing the proliferation of fibroblasts, and that right circularly polarized light increased the expression of type 1 procollagen mRNA. They hypothesized that the effectiveness of right circularly polarized light in wound healing suggests that an optically active material, having a circular dichroic spectrum, takes part in a biochemical reaction underlying the process. [The Tada K, Ikeda K, Tomita K. Effect of polarized light emitting diode irradiation on wound healing. J Trauma. 2009; 67(5):1073-1079].

While the use of polarized light in light therapy may have some notable advantages, little consideration has been given to the manner in which polarized light may be manipulated, especially in therapeutic applications.

It has now been found that beneficial effects may be obtained, especially in electromagnetic radiation therapy or light therapy applications (including, but not limited to, brainwave entrainment), by oscillating electromagnetic radiation between two or more polarization states. This may include, for example, oscillating electromagnetic radiation between nonpolarized and polarized states, oscillating electromagnetic radiation between distinct polarization states (as, for example, by oscillating electromagnetic radiation between right-handed and left-handed polarization states), oscillating electromagnetic radiation between types of polarization (as, for example, by oscillating electromagnetic radiation between two or more of circularly polarized, elliptically polarized or linearly polarized states), or oscillating electromagnetic radiation between two or more distinct planes of polarization (as, for example, by oscillating electromagnetic radiation between at least two polarization states in which the electromagnetic radiation has an electric field confined, respectively, to first and second planes that are non-coplanar).

Various combinations of the foregoing approaches may also be utilized. For example, embodiments are possible in which oscillation of electromagnetic radiation occurs between two or more states which differ in at least two parameters selected from the group consisting of polarized/nonpolarized, orientation of polarization, type of polarization, and plane of polarization.

Illumination or light therapy devices may be made in accordance with the teachings herein which feature one or more LED arrays. In some embodiments, the one or more LED arrays may contain distinct groups of LEDs, one or more of which may oscillate among two or more polarization states. For example, in some embodiments, a first group may be non-oscillatory or may oscillate between an on and off state, while one or more additional groups of LEDs may oscillate between two or more polarization states. In other embodiments, each group of LEDs may oscillate among a unique set of polarization states.

In still other embodiments, the LED array may include at least a first and second set of LEDs, wherein the first set of LEDs emits light of a first wavelength or range of wavelengths, wherein the second set of LEDs emits light of a second wavelength or range of wavelengths which is the same as or different from the first wavelength or range of wavelengths, and wherein at least one (and in some embodiments, both) of the first and second sets of LEDs oscillates among two or more polarization states. For example, in one such embodiment, a first set of LEDs emits non-oscillating red light and a second set of LEDs emits red light which oscillates between first and second polarization states. In another such embodiment, a first set of LEDs emits red light which oscillates between a first and second polarization state, and a second set of LEDs emits blue light which oscillates between third and fourth polarization states. In a further such embodiment, a first set of LEDs emits red light which oscillates between a first and second polarization state, a second set of LEDs emits blue light which oscillates between third and fourth polarization states, and a third set of LEDs emits green light which oscillates between a fifth, sixth and seventh polarization states.

The oscillation frequency between polarization states of electromagnetic radiation may be selected to achieve various results. For example, in some non-limiting embodiments of the devices and methodologies disclosed herein which relate to light therapy applications, this oscillation frequency may be selected to entrain brainwaves, preferably in one or more of the five common brainwave bandwidths (delta, theta, alpha, beta and gamma). In these and other applications, the electromagnetic radiation or the periodicity thereof may be synchronized to sound waves or beats, pressure waves, electrical stimulation, vibration or touch therapy. These include, without limitation, binaural beats and sound having nested wave functions or nested frequencies of the type described in aforementioned PCT/US21/42675 (Fortkort et al.).

It has further been found that beneficial effects may be obtained, especially in light therapy applications (including, but not limited to, those involving brainwave entrainment), by utilizing a composite waveform constructed from component waveforms having distinct polarization states. These polarization states may include, for example, nonpolarized and polarized states, polarization states characterized by distinct orientations of polarization (for example, right-handed and left-handed polarization), distinct types of polarization (for example, circularly polarized, elliptically polarized or linearly polarized states), or distinct planes of polarization (for example, first and second polarization states in which the electromagnetic radiation has an electric field confined to first and second distinct or non-coplanar states, respectively).

Various combinations of the foregoing may also be utilized. For example, composite waveforms may be constructed from waveforms whose polarizations differ in at least two parameters selected from the group consisting of polarized/nonpolarized, orientation of polarization, type of polarization, and plane of polarization. The oscillation frequencies of the composite waveforms having distinct polarized states may be selected, for example, to entrain brainwaves in one or more frequency regimes (as, for example, in one or more of the five common brainwave bandwidths (delta, theta, alpha, beta and gamma)). In some embodiments, the composite waveform may include multiple component waveforms which entrain at multiple distinct frequencies. The foregoing composite waveforms may be used in conjunction with other techniques, such as binaural beats, sound waves, pressure waves, electrical stimulation, vibration or touch therapy.

Prior to describing specific embodiments of the devices and methodologies disclosed herein in greater detail, it is to be noted that all references to polarization herein utilize the convention of being from the point of view of the source. In accordance with this convention, left-handedness or right-handedness is determined by pointing one's left or right thumb away from the source of electromagnetic radiation and in the direction that the wave is propagating in, and matching the curling of one's fingers to the direction of the temporal rotation of the field at a given point in space. If this operation requires use of the right hand, then the electromagnetic radiation is polarized in a right-handed polarization state. Similarly, if this operation requires use of the left hand, then the electromagnetic radiation is polarized in a left-handed polarization state.

An analogous approach is utilized in determining whether the wave is polarized in a clockwise or counter-clockwise manner. Here, one again takes the point of view of the source of electromagnetic radiation, and while looking away from the source and in the same direction of the wave's propagation, one observes the direction of the field's spatial rotation. If the rotation viewed from this perspective is clockwise, then the electromagnetic radiation is polarized in a clockwise polarization state. Similarly, if the rotation viewed from this perspective is counterclockwise, then the electromagnetic radiation is polarized in a counterclockwise polarization state.

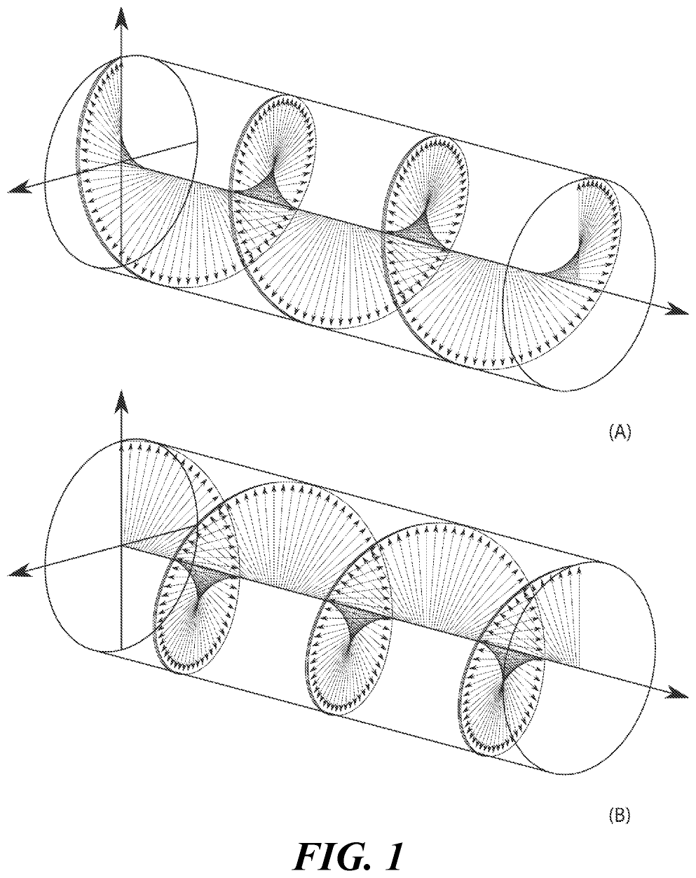

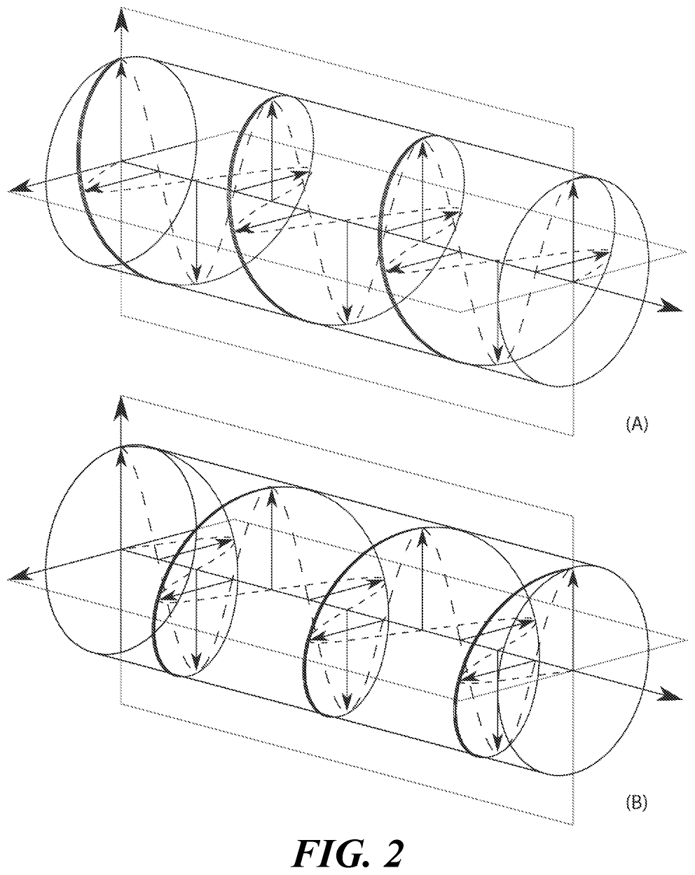

FIGS. 1-2 illustrate the concept of circularly polarized light. In FIGS. 1A and 2A, the electromagnetic radiation is polarized in a right-handed or clockwise polarization state, while in FIGS. 1B and 2B, the electromagnetic radiation is polarized in a left-handed or counterclockwise polarization state. FIG. 1 depicts the vector of the electromagnetic radiation as it propagates along an axis, while FIG. 2 depicts both the vector and the component electric and magnetic fields of the electromagnetic radiation as it propagates along an axis. It will be appreciated from FIGS. 1 and 2 that there is a first plane containing the axis of propagation of the electromagnetic radiation to which the electric field of the electromagnetic radiation is confined. Similarly, it will be appreciated that there is a second plane containing the axis of propagation of the electromagnetic radiation to which the magnetic field of the electromagnetic radiation is confined. It will further be appreciated that these first and second planes are mutually orthogonal. As the electromagnetic radiation travels along its axis of propagation, the vector of the electric and magnetic fields traces out a circle. The direction of movement of this vector is right-handed or clockwise in FIGS. 1A and 1B and is left-handed or counterclockwise in FIGS. 1B and 2B. Hence, polarized electromagnetic radiation having this quality is referred to as being “circularly polarized”, with the polarization of FIGS. 1A and 2A being referred to as being in a “right-handed”or clockwise polarization state, and the polarization of FIGS. 1B and 2B being referred to as being in a “left-handed” or counterclockwise polarization state.

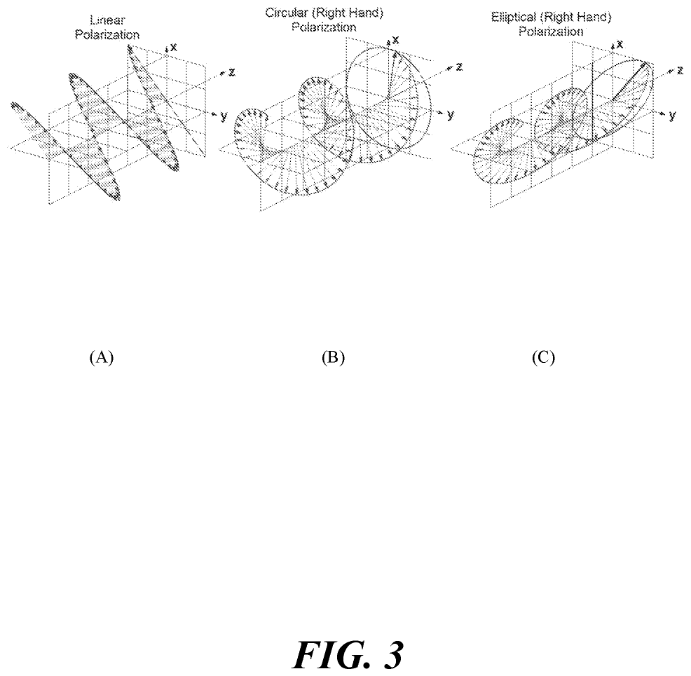

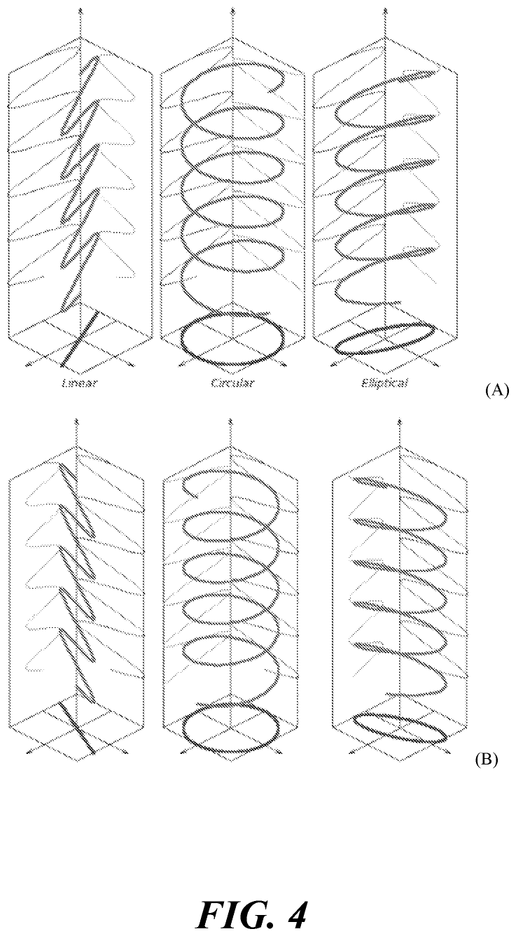

The concept of elliptical polarization is wholly analogous to circular polarization. It will be appreciated from FIGS. 3 and 4 that, in elliptical polarization, there is a first plane containing the axis of propagation of the electromagnetic radiation to which the electric field of the electromagnetic radiation is confined. Similarly, it will be appreciated that there is a second plane containing the axis of propagation of the electromagnetic radiation to which the magnetic field of the electromagnetic radiation is confined. It will further be appreciated that these first and second planes are mutually orthogonal. As the electromagnetic radiation travels along its axis of propagation, the vector of the electric and magnetic fields traces out an ellipse. The direction of movement of this vector is right-handed or clockwise in FIG. 4B, and is left-handed or counterclockwise in FIG. 4A. Hence, polarized electromagnetic radiation having this quality is referred to as being “elliptically polarized”, with the polarization of FIG. 4A being referred to as being in a “right-handed” or clockwise polarization state, and the polarization of FIG. 4B being referred to as being in a “left-handed” or counterclockwise polarization state.

It will be appreciated that linear and circular polarization are special cases of elliptical polarization. The classical sinusoidal plane wave solution of the electromagnetic wave equation for the electric and magnetic fields of polarized electromagnetic radiation (in Gaussian units) is given by:

E(r,t)=|E|Re{|φ [symbol] exp[i(kz−ωt)}

B(r,t)=\hat{z}×E(r,t) (EQUATION 1)

for the magnetic field, where k is the wavenumber,

ω=ck (EQUATION 2)

is the angular frequency of the wave propagating in the +z direction, and c is the speed of light. In EQUATION 1, |E| is the amplitude of the field, and the normalized Jones vector |φ> is defined by EQUATION 3:

$$\vert φ 〉 {=}^{def} ( \begin{matrix}{φ}_{x} \\ {φ}_{y}\end{matrix} ) = ( \begin{matrix}cos θ exp ( {ia}_{x} ) \\ sin θ exp ( {ia}_{y} )\end{matrix} ) \qquad ( EQUATION 3 )$$

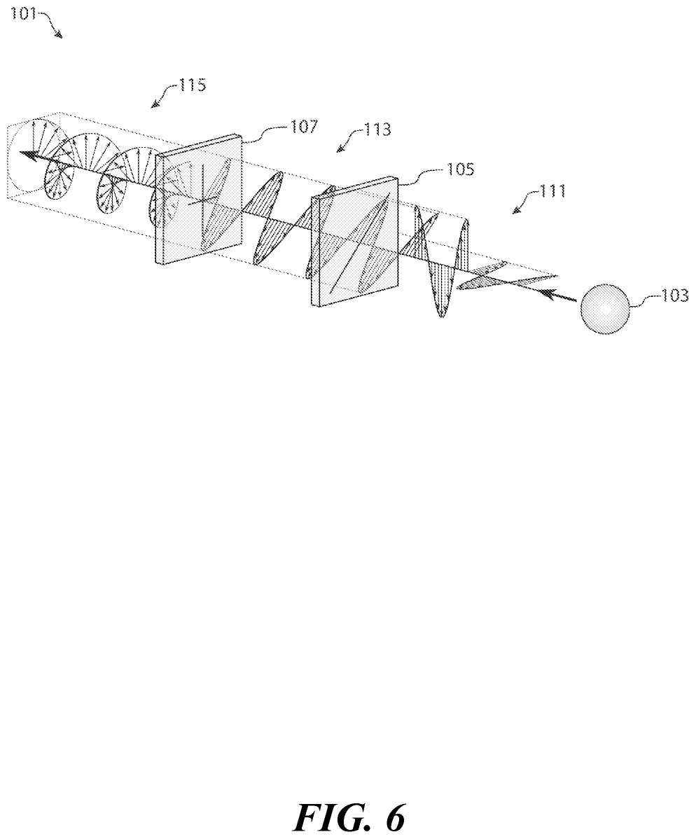

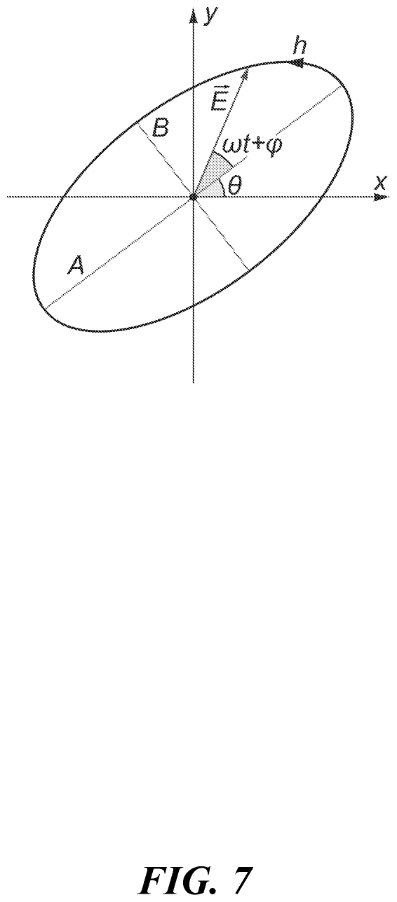

At a fixed point in space (or for fixed z), the electric vector E traces out an ellipse in the x-y plane with semi-major and semi-minor axes of lengths A and B, respectively (see FIG. 6). The lengths of these axes are given by EQUATIONS 4 and 5:

$$A = \vert E \vert \sqrt{\frac{1 + \sqrt{1 - {sin}^{2} ( 2 θ ) {sin}^{2} β}}{2}} \qquad ( EQUATION 4 )$$

$$B = \vert E \vert \sqrt{\frac{1 - \sqrt{1 - {sin}^{2} ( 2 θ ) {sin}^{2} β}}{2}} \qquad ( EQUATION 5 )$$

wherein β=α_{y}−αₓ. The orientation of the foregoing ellipse may be described in terms of the angle between the semimajor axis of the ellipse and the x-axis. This angle may be derived from EQUATION 6:

tan 2ø=tan 2θ cos β (EQUATION 6)

It follows from the foregoing that, in the special case when β=0, the ellipse collapses into a single line, and the wave is thus linearly polarized.

When β≠0, then A≠B, and the wave is elliptically polarized. In particular, when β is positive, an ellipse is traced out in the counterclockwise direction (when viewed in the direction of the propagating wave), and the wave is in a left-handed elliptical polarization state. When β is negative, an ellipse is traced out in the clockwise direction, and the wave is in a right-handed elliptical polarization state.

In the special case where β=±π/2 and θ=±π/4, then A=B=|E|/\sqrt{2′} and the wave will be circularly polarized. Here, the direction of polarization is determined by the sign of β. Thus, if β=π/2, the wave will be left-circularly polarized, and β=−π/2, the wave will be right-circularly polarized.

Various methods may be utilized to impart polarization to electromagnetic radiation in the devices and methodologies disclosed herein. These include, but are not limited to, polarization by reflection, refraction, scattering, transmission, or various combinations of the foregoing. Each of these techniques for imparting polarization to electromagnetic radiation is described in greater detail below.

Polarization by reflection utilizes (typically non-metallic) surfaces to polarize incident electromagnetic radiation through reflection from such surfaces. The amount of polarization may depend on such factors as the composition of the surface and the angle of the incident electromagnetic radiation. While metallic surfaces often reflect electromagnetic radiation with a variety of unpolarized vibrational directions, non-metallic surfaces frequently reflect electromagnetic radiation such that there will be a large concentration of vibrations in a plane parallel to the reflecting surface.

Polarization by refraction uses the principal of refraction (that is, a change in direction of electromagnetic radiation as it passes from one medium to another, or as it passes through a medium having an index of refraction that undergoes changes along the path of the electromagnetic radiation) to achieve polarization. If light is incident upon the surface of a suitable material such as glass, then part of the light will be refracted, and the other part will be reflected. This process imparts polarization to the incident light such that the vector of the electrical field strength of the polarized reflected light oscillates at right angles to the incident plane, and that of the refracted light oscillates parallel to the incident plane. Polarization of the refracted light will increase as the amount by which the angle of incidence deviates from 56° decreases. Polarization of the refracted light will also increase as the light passes through more refractive surfaces of this type.

Polarization by scattering uses the scattering of electromagnetic radiation to induce polarization. In particular, when electromagnetic radiation is incident on a material containing particles, molecules or other optically scattering centers, a portion of the incident electromagnetic radiation is a scattered in various directions. The scattering may be forward scattering, backward scattering, or both, and the scattering may be anisotropic as a function of scattering angle with respect to its polarization. For example, in some cases, the portion of the electromagnetic radiation which is forward scattered in the direction perpendicular to the incident electromagnetic radiation will be completely polarized, while the portion of the electromagnetic radiation which is not scattered (that is, which undergoes transmission along the original axis of incidence) will be unpolarized. The portion of the electromagnetic radiation which is forward scattered in directions between these extremes will be partially polarized.

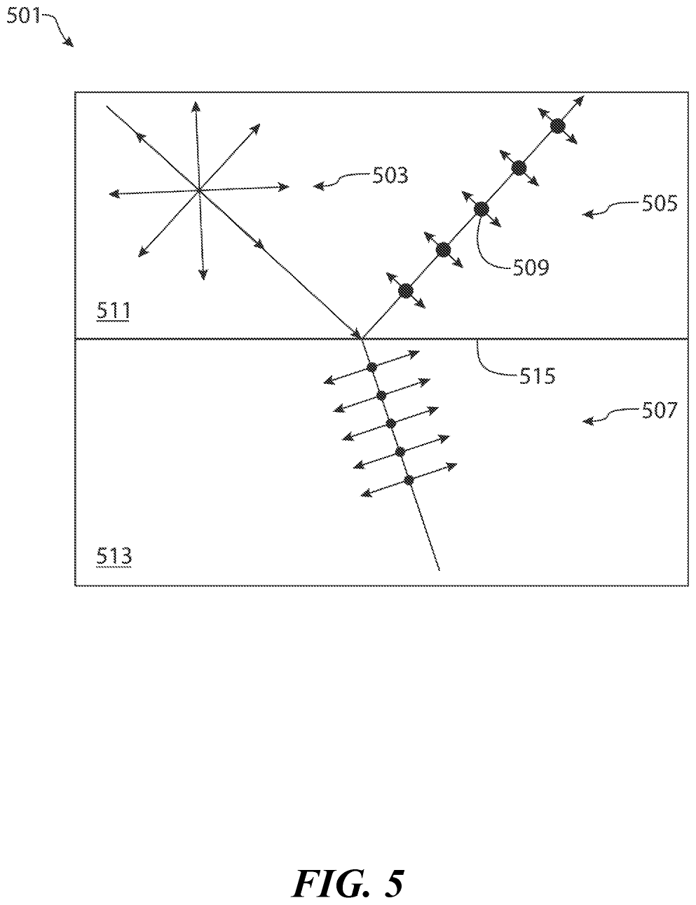

FIG. 5 illustrates polarization by refraction and polarization by reflection. As seen in the system 501 depicted therein, when unpolarized light 503 impinges upon an interface 515 between two materials (here, air 511 and glass 513) having different refractive indices, a portion of the light 505 is reflected, and a portion of the light 507 is refracted (note that the circles 509 represent arrows which are perpendicular to the page). The reflected 505 and refracted 507 portions of light will each be partially polarized. It will be appreciated that multiples of such reflections and/or transmissions may be utilized to polarize electromagnetic radiation to varying degrees of polarization.

The foregoing principles may be implemented in various ways to produce highly polarized light. Thus, for example, birefringent multilayer optical films may be produced in which the refractive indices in the thickness direction of two adjacent layers are substantially matched have a Brewster angle (the angle at which reflectance of p-polarized light goes to zero) which is very large or is nonexistant. This allows for the construction of multilayer mirrors and polarizers whose reflectivity for p-polarized light decreases slowly with angle of incidence, are independent of angle of incidence, or increase with angle of incidence away from the normal. As a result, multilayer films having high reflectivity (for both planes of polarization for any incident direction in the case of mirrors, and for the selected direction in the case of polarizers) over a wide bandwidth, may be achieved. Multilayer optical films of this type are described, for example, in U.S. Pat. No. 5,882,774 (Jonza et al.), which is incorporated herein by reference in its entirety. It will be appreciated that the multilayer optical films of Jonza et al. may thus be utilized to polarize incident electromagnetic radiation through reflection or transmission.

The foregoing principles may also be implemented in various ways to produce highly polarized light by scattering. This may be achieved, for example, through the use of continuous/disperse phase optical films of the type disclosed in U.S. Pat. No. 6,031,665 (Carlson et al.) and U.S. Pat. No. 6,654,170 (Merrill et al.), both of which are incorporated herein by reference in their entirety. These optical films comprise a disperse phase of polymeric particles disposed within a continuous birefringent matrix. The film is oriented, typically by stretching, in one or more directions. The size and shape of the disperse phase particles, the volume fraction of the disperse phase, the film thickness, and the amount of orientation may be chosen to attain a desired degree of diffuse reflection and total transmission of electromagnetic radiation of a desired wavelength in the resulting film.

FIG. 6 depicts a first particular, non-limiting embodiment of a device which may be utilized in some embodiments of the devices disclosed herein to convert randomly polarized electromagnetic radiation into polarized electromagnetic radiation. As seen therein, the device 101 comprises a source 103 of randomly polarized radiation. A linear polarizer 105 is disposed in the optical path of the source 103 and functions to convert impinging, randomly polarized electromagnetic radiation 111 into linearly polarized electromagnetic radiation 113. A quarter-wave plate 107 is provided in the optical path of the linearly polarized electromagnetic radiation which converts it into circularly polarized light 115. In the particular embodiment depicted, the quarter-wave plate 107 imparts left-handed polarization to the linearly polarized electromagnetic radiation, although embodiments are also possible in which the quarter-wave plate 107 imparts right-handed polarization to the linearly polarized electromagnetic radiation. It will also be appreciated that the quarter wave plate 107 may be dispensed with in applications requiring linearly polarized electromagnetic radiation.

It will be appreciated from FIG. 6 that linear polarizers of varying orientations may be utilized to produce linearly polarized electromagnetic radiation from incident, randomly polarized electromagnetic radiation. These linear polarizers may operate through reflection, dichroism or double refraction/birefringence.

Various types of double refraction or birefringent polarizers may be utilized to produce linearly polarized light from randomly polarized light in the devices and methodologies disclosed herein. These include, for example, the use of crystals of calcite and quartz, which are capable of dividing a single impinging and randomly polarized beam into two separate, polarized beams of equal intensity. In some cases, these polarizers act as beam-splitting polarizers to divide incident electromagnetic radiation into two orthogonal, linearly polarized beams. In some embodiments, one beam will be transmitted at the original angle of incidence, and the other beam will be reflected at an angle orthogonal to the original angle of incidence. Such a splitting may be especially advantageous in some applications. In various embodiments, these polarizers may be equipped with low absorption coatings to improve damage resistance or modify the extinction ratio. In some cases, the extinction ratio of the reflected beam may be improved through the provision of a dichroic polarizer to the output surface for that beam.

Various types of reflection polarizers may be utilized to produce linearly polarized light from randomly polarized light in the devices and methodologies disclosed herein. Preferably, these polarizers will feature a flat, smooth and non-metallic reflective surface. When a randomly polarized beam of electromagnetic radiation strikes such a surface at a suitable angle, the reflected beam will be partially or completely polarized, with the degree of polarization typically depending on the angle of incidence and the refractive index of the reflecting surface (and more specifically, the difference in the refractive indices of the reflective surface and the ambient medium through which the electromagnetic radiation is propagating). The angle at which the degree of polarization is 100% is referred to as the Brewster's angle.

Various types of dichroic polarizers may be utilized to produce linearly polarized light from randomly polarized light in the devices and methodologies disclosed herein. These polarizers exhibit dichroism (that is, they absorb light that is polarized in a particular direction). Hence, dichroic linear polarizers have an absorption and transmission axis, the latter of which is referred to as the “polarizing axis.” Various materials may be utilized to produce linear polarizers including, for example, oriented Polyvinyl Alcohol (PVA).

Various other polarizers, which may belong to one or more of the types noted above, may be utilized in the devices and methodologies disclosed herein. These include, without limitation, those which utilize Glan-Thompson, Nicol, Glan-Foucault or Glan-Taylor prisms or beam splitters. Glan-Thompson polarizers may be fabricated, for example, from two right-handed calcite prisms cemented together along their long faces. The cement utilized for this purpose may be a suitable synthetic polymer or Canada balsam.

Various lenses may be utilized to focus or otherwise manipulate electromagnetic radiation in the devices and methodologies disclosed herein. These include, without limitation, simple, compound and Fresnel lenses. These lenses may be biconvex, plano-convex, plano-concave, biconcave, or may be lenses having a positive or negative meniscus. For example, one or more of the foregoing lenses may be utilized to focus the rays of multiple light sources (such as, for example, multiple LEDs) onto a single focal point. The use of such lenses may facilitate mixing of electromagnetic radiation from distinct sources which may emit electromagnetic radiation at distinct wavelengths or states of polarization. The use of such lenses may also facilitate manipulation of the polarization state of incident electromagnetic radiation.

Some embodiments of the devices and methodologies disclosed herein may make advantageous use of switchable waveplates, including birefringent rotators. Half-wave plates and quarter-wave plates utilize the principle of birefringence to alter the polarization of incident light, an effect which may be wavelength-specific. However, switchable waveplates may be utilized to rapidly change the angle of polarization of incident electromagnetic radiation in response to an electric signal, and can therefore be used for rapid polarization state generation (PSG). Hence, these devices may be utilized in the devices and methodologies disclosed herein to oscillate the polarization of light from an LED or other light source. Switchable wave plates suitable for use in the devices and methodologies disclosed herein may be fabricated from various materials including, but not limited to, liquid crystals, ferro-electric liquid crystals, or magneto-optic crystals.

As a specific example of the foregoing, half-wave retarders may be utilized in the systems and methodologies disclosed herein which feature a stack of one nematic liquid-crystal cell with uniform alignment sandwiched between two twisted nematic layers that have identical twist angles (e.g., 135°) but different orientations of their surface alignment. The resulting device may be utilized as an optical switch for light with linear polarization at 45° to the optic axis of the homogeneous cell. In particular, in the absence of an electric field, this switch may function to rotate incident linear polarization by 90 degrees, while in the presence of a suitable electric field (and in particular, when sufficient voltage is applied to all three layers of the device), the switch may induce little or no change in the polarization of incident electromagnetic radiation. Switches of this type have been demonstrated which exhibit an achromatic response in the spectral range 400-700 nm for both activated and quiescent states. [See, e.g., M. Lavrentovich, T. Sergan, and J. Kelly, “Switchable broadband achromatic half-wave plate with nematic liquid crystals,” Opt. Lett. 29, 1411-1413 (2004)].

Electrically switchable waveplates may also be utilized in the devices and methodologies disclosed herein, and these waveplates may utilize diffractive waveplates or their equivalent metasurfaces. Metasurfaces (two-dimensional artificially engineered media containing thin optical resonators of different materials and geometries) may be utilized to manipulate the amplitude and/or phase of electromagnetic radiation. Such metasurfaces may be utilized alone or in combination with tunable liquid crystals or phase change materials (such as, for example, vanadium dioxide (VO₂)). Switchable waveplates of this type are described, for example, in [J. Chou, L. Parameswaran, B. Kimball, and M. Rothschild, “Electrically switchable diffractive waveplates with metasurface aligned liquid crystals,” Opt. Express 24, 24265-24273 (2016)] and in [Wang, D., Zhang, L., Gu, Y. et al. Switchable Ultrathin Quarter-wave Plate in Terahertz Using Active Phase-change Metasurface. Sci Rep 5, 15020 (2015); Hao, J. M. et al. Optical metamaterial for polarization control. Phys. Rev. A 80, 023807 (2009); Ma, X. L. et al. Dual-band asymmetry chiral metamaterial based on planar spiral structure. Appl. Phys. Lett. 101, 161901 (2012); and Pu, M. B. et al. Anisotropic meta-mirror for achromatic electromagnetic polarization manipulation. Appl. Phys. Lett. 102, 131906 (2013)].

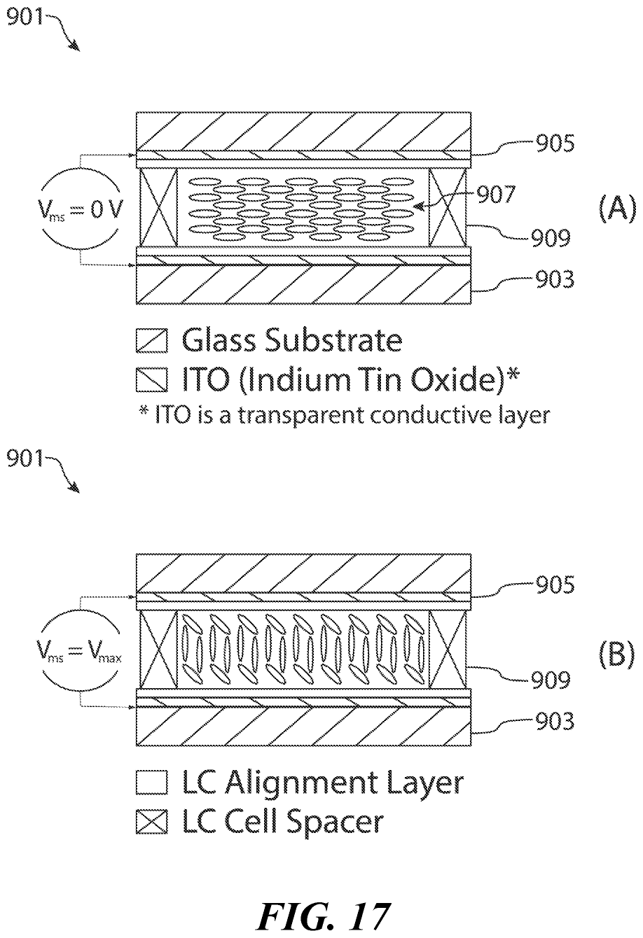

FIG. 17 depicts a particular, non-limiting embodiment of an electrically switchable device of the foregoing type. In the particular embodiment depicted, the device 901 comprises a glass substrate 903 with a, indium tin oxide (ITO) layer 905 disposed thereon. A liquid crystal (LC alignment layer 907 and LC cell spacer 909 are disposed between the ITO layers 905. As shown in FIG. 17A, in their nematic phase, the liquid crystal molecules have an ordered orientation which, in combination with the stretched shape of the molecules, generates optical anisotropy. When an electric field is applied (FIG. 17B), the molecules align to the field, and the level of birefringence may be controlled by the tilting of the LC molecules. Hence, by appropriately oscillating the applied electric field, devices of this type may be utilized to generate a light source which oscillates between two or more polarization states.

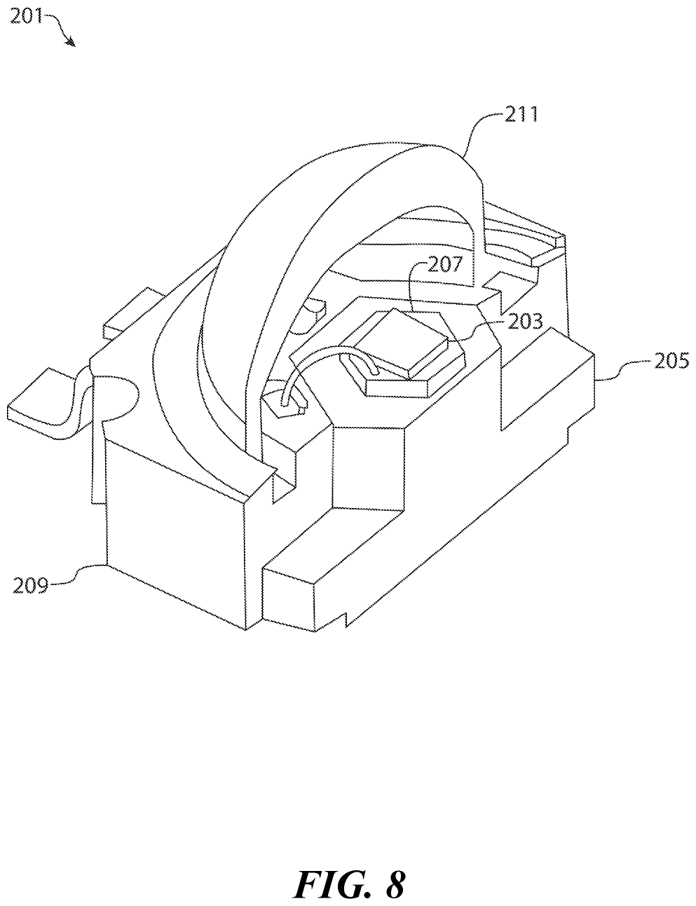

Various types of LEDs may be utilized as the source for electromagnetic radiation in the devices and methodologies disclosed herein. FIG. 8 depicts a particular, non-limiting embodiment of such an LED. The LED 201 depicted therein comprises a chip or die 203 attached to a heat sink 205 by way of a bonding substrate 207. The die 203 and heat sink 205 are housed in an outer package 209. A lens or other primary optic 211 is provided to impart primary optical characteristics to the electromagnetic radiation emitted by the die 203. It will be appreciated that the die 203 may include multiple light-emitting regions, and may be an LED array.

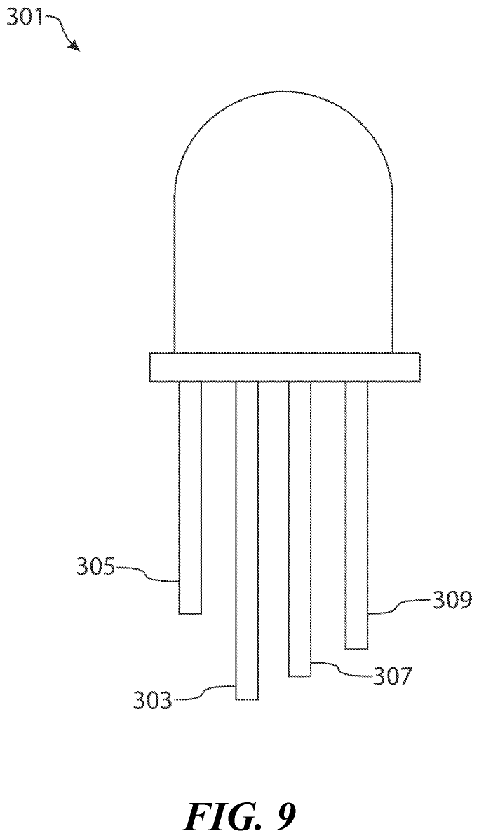

FIG. 9 depicts an LED 301 which may be the same as, or different from, the LED of FIG. 8, and which may be utilized in some of the devices and methodologies disclosed herein. The particular LED 301 depicted is an RGB LED with a common anode 303, and grounded pins 305, 307 and 309 for the cathode terminals of the green, blue and red LEDs, respectively. In the particular embodiment depicted, the common anode carries a voltage of +3V.

In operation, pin 303 is connected to +3V of power, which powers all of the LEDs. A series of toggle switches (not shown) are then connected to positive voltage and ground, thus allowing the individual LEDs to be turned off or on. When each toggle switch is flipped to the ground terminal side, the LED turns on, and when it is switched to the +3V terminal side, the LED turns off.

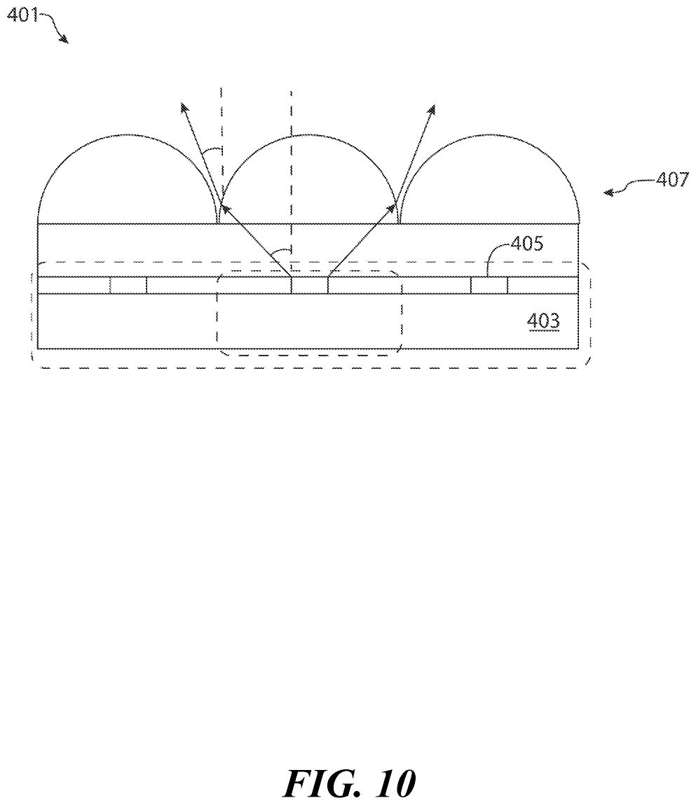

FIG. 10 depicts a first embodiment of a source of electromagnetic radiation which may be utilized in the devices and methodologies disclosed herein.

Some embodiments of the devices and methodologies disclosed herein may utilize an LED array. A particular, non-limiting embodiment of a source of electromagnetic radiation incorporating such an LED array is shown in FIG. 10. The light source 401 depicted therein includes a substrate 403 upon which is disposed a plurality of pixel light sources 405. The pixel light sources 405 are preferably LEDs. A micro lens array 407 is disposed over, and in the optical path of, the pixel light sources 405, and is physically separated therefrom by way of an optical spacer 409. The micro lens array 407 is preferably positioned such that the individual lens elements thereof are centered over the pixel light sources 405. As depicted in FIG. 10, the micro lens array 407 refracts the electromagnetic radiation emitted by the pixel light sources 405, thus narrowing the diameter of the light cones emitted by the pixel light sources 405.

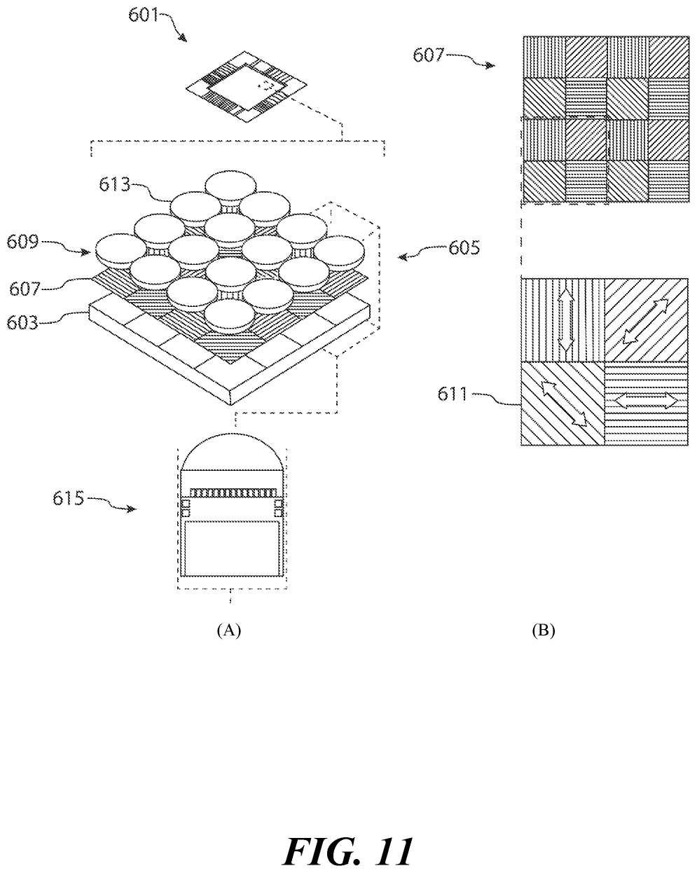

FIG. 11 depicts a further embodiment of a source 601 of electromagnetic radiation which may be utilized in some embodiments of the devices and methodologies disclosed herein. In the particular embodiment depicted, the source 601 of electromagnetic radiation includes a substrate 603 having an LED array 605 disposed thereon. A polarizer array 607 and micro lens array 609 are disposed over (and in the optical path of) the LED array 605. The polarizer array 607 contains a plurality of individual polarizing elements 611, each of which is preferably centered over an LED in the LED array 605. Similarly, the micro lens array 609 contains a plurality of individual lens elements 613, each of which is preferably centered over an LED 615 in the LED array 605. In some variations of this embodiment, one or more optical spacers or other optical elements may be disposed between the LED array 605 and the polarizer array 607 or between the polarizer array 607 and the micro lens array 609.

In the particular embodiment depicted, the polarizer array 607 imparts one or four distinct linear polarization states to the electromagnetic radiation emitted by each of the LEDs 615 in the LED array 605. The individual LEDs 615 in the LED array 605 may thus be operated (for example, turned on and off) in such a manner that the polarization of the electromagnetic radiation emitted by the light source oscillates in a desired manner between two or more of these polarization states. Thus, for example, the individual LEDs 615 in the LED array 605 may be activated such that the source of electromagnetic radiation 601 oscillates (e.g., turns on and off) at a frequency of 40 Hz, and such that the polarization of emitted radiation rotates by 45° with each oscillation. This will effectively produce an output of electromagnetic radiation which oscillates at 10 Hz with respect to any particular polarization.

Of course, it will be appreciated that several variations of the foregoing embodiment are possible. For example, in some embodiments, the polarizer array may contain only two or three types of polarizers. It will also be appreciated that any of the polarizers in the polarizer array may be independently selected from the group consisting of linear, circular or elliptical polarizers, or that some of the polarizers in the polarizer array may be replaced with nonpolarizing elements. It will further be appreciated that the optical properties of each polarizer in the polarizer array may be selected to achieve a desired pattern in the footprint of the source of electromagnetic radiation.

In some variations of the foregoing embodiment, the polarizer array may include a first set of optical elements which impart a first polarization state to incident electromagnetic radiation, and a second set of optical elements which either do not change the polarization state of incident electromagnetic radiation, or which randomize the polarization of incident electromagnetic radiation. First and second sets of LEDs corresponding, respectively, to the first and second sets of optical elements may then be activated in various sequences to produce desired results. For example, the first and second sets of LEDs may be activated in an alternating, periodic manner to produce an output of electromagnetic radiation that oscillates (between on/off states) at a first frequency (for example, 40 Hz), and which oscillates between polarized/nonpolarized states at a second frequency (e.g., 20 Hz).

Analogous embodiments are possible in which the polarizer array may include a first set of optical elements which impart a first polarization state to incident electromagnetic radiation, and a second set of optical elements which impart a second polarization state to incident electromagnetic radiation. First and second sets of LEDs corresponding, respectively, to the first and second sets of optical elements may then be activated in various sequences to produce desired results. For example, the first and second sets of LEDs may be activated in an alternating, periodic manner to produce an output of electromagnetic radiation that oscillates (between on/off states) at a first frequency (for example, 40 Hz), and which oscillates between first and second polarization states at a second frequency (e.g., 20 Hz). This may include, for example, oscillating the electromagnetic radiation between distinct orientations of polarization (for example, oscillating electromagnetic radiation between right-handed and left-handed polarization states), oscillating electromagnetic radiation between types of polarization (for example, oscillating electromagnetic radiation between two or more of circularly polarized, elliptically polarized or linearly polarized states), or oscillating electromagnetic radiation between at least two distinct planes or polarization (for example, oscillating electromagnetic radiation between at least two polarization states in which the electromagnetic radiation has an electric field confined to first and second non-coplanar planes, respectively).

One skilled in the art will appreciate that variations of the foregoing embodiments are possible in which the selection of individual polarizers in the polarizer array, selection of the frequency at which individual LEDs in the LED array are activated (which may, in some embodiments, be varied as a function of time), or selection of the wavelengths of electromagnetic radiation emitted by the individual LEDs in the LED array, may be utilized to produce a wide variety of outputs of electromagnetic radiation from the source of electromagnetic radiation. Moreover, the polarization state (or states) of this output, the wavelengths of the output, and the frequency at which a given wavelength oscillates between two or more polarization states, may vary over time.

Various types of secondary optics may be utilized to manipulate electromagnetic radiation in the devices and methodologies disclosed herein. These include, without limitation, various reflectors, diffusers, and polarizers, any of which may be specularly or diffusely transmitting or reflecting or color-shifting. Such secondary optics may be utilized, for example, to modify the angle or beam shape of electromagnetic radiation produced by one or more sources.

In some embodiments of the devices and methodologies disclosed herein, polarizing filters may be utilized that feature an oscillating refractive index that produces light whose polarization thus oscillates between first and second states. Such a result may be achieved, for example, through the use of a filter material whose refractive index may be manipulated with a magnetic field that may itself be oscillated. Suitable materials of this type include, without limitation, La_{0.66}Sr_{0.33}MnO₃ [see Strutner, Scott & Garcia, Adam & Ula, Sabina & Adamo, Carolina & Richards, W. Lance & Wang, Kang & Schlom, Darrell & Carman, Greg. (2017). Index of refraction changes under magnetic field observed in La_066Sr_033MnO_3 correlated to the magnetorefractive effect. Optical Materials Express. 7. 468. 10.1364/OME.7.000468].

Some embodiments of the devices and methodologies disclosed herein may utilize birefringent films to generate light with an oscillating polarization state. Such birefringence may be uniaxial or biaxial. Birefringent films of this type may include, for example, any of the films disclosed in previously noted U.S. Pat. No. 5,882,774 (Jonza et al.), U.S. Pat. No. 6,031,665 (Carlson et al.) or U.S. Pat. No. 6,654,170 (Merrill et al.).

In uniaxial birefringent materials, the optical anisotropy occurs in a single direction (the optical axis), while all directions perpendicular to the optical axis (or at a given angle to it) are optically equivalent. Light propagating parallel to the optical axis (whose polarization is perpendicular to the optic axis) is governed by an “ordinary” refractive index nₒ, regardless of its specific polarization. For light traveling along any other propagation direction, one linear polarization exists perpendicular to the optical axis, and light with that polarization is governed by the same refractive index value no. A ray of this type is referred to as an “ordinary ray”. For any ray propagating in the same direction but with a polarization perpendicular to that of the ordinary ray (such a ray is referred to as an extraordinary ray”), the polarization direction will be partly in the direction of the optical axis, and the refractive index experienced by that ray will be direction-dependent. Because the index of refraction for unpolarized incident radiation depends on its polarization when it enters a uniaxial birefringent material, the incident unpolarized radiation is split into two beams that travel in different directions. One of these beams has the polarization of the ordinary ray, and the other beam has the polarization of the extraordinary ray. The ordinary ray will always experience a refractive index of no, while the extraordinary ray will experience a refractive index between nₒ and nₑ, depending on the ray direction as described by the index ellipsoid. The magnitude of the difference is quantified by the birefringence in accordance with EQUATION 7:

Δn=nₑ−nₒ (EQUATION 7)

The propagation (and reflection coefficient) of the ordinary ray is described by no (as if there were no birefringence involved). However, the extraordinary ray propagates in a manner that is notably different from the propagation of a wave in an isotropic material (hence the name). The refraction (and reflection) of such a ray at a surface can be understood using the effective refractive index (a value in between nₒ and nₑ). However, its power flow (which is described by the Poynting vector) differs from the direction of the wave vector. This causes an additional shift in that beam, even when launched at normal incidence.

When the light propagates along (or orthogonal to) the optical axis, the foregoing lateral shift does not occur. In the first case, both polarizations are perpendicular to the optic axis, and thus see the same effective refractive index (hence, no extraordinary ray exists). In the second case, the extraordinary ray propagates at a different phase velocity (corresponding to nₑ), but retains a power flow in the direction of the wave vector. A crystal with its optic axis in this orientation, parallel to the optical surface, may thus be used to create a waveplate in which the state of polarization of the incident wave is modified. For example, a quarter-wave plate may be utilized to generate circularly polarization light from linearly polarized light.

The situation with biaxial birefringent crystalline materials is significantly more complex. Such materials are characterized by three refractive indices which correspond to the three principal axes of the crystal. For most ray directions, both polarizations may be classified as extraordinary rays having different effective refractive indices. Since both are extraordinary rays, however, the direction of power flow is not identical to the direction of the wave vector. The refractive indices experienced by these rays may be determined using the index ellipsoids corresponding to given directions of the polarization. For biaxial crystalline materials, the index ellipsoid will not be an ellipsoid of revolution (“spheroid”) but will be described by three unequal principle refractive indices n_{α}, n_{β} and n_{γ}. Consequently, no rotational axis of symmetry exists around which the optical properties of the material are invariant. However, two optical axes or binormals exist which are defined as directions along which light may propagate without birefringence (that is, directions along which the wavelength is independent of polarization). Hence, birefringent materials with three distinct refractive indices are referred to as biaxial materials. Additionally, two distinct axes (termed “optical ray axes” or “biradials”) exist along which the group velocity of the light is independent of polarization.

Some embodiments of the devices and methodologies disclosed herein may utilize groups of LEDs to generate electromagnetic radiation with an oscillating polarization state. In preferred embodiments of this type, at least one member of each group of LEDs is an LED which emits electromagnetic radiation in a first polarization state, while at least one member of each group of LEDs electromagnetic radiation light in a second polarization state. For example, the first polarization state may be unpolarized, and the second polarization state may be polarized. Examples of LEDs which emit polarized electromagnetic radiation include, but are not limited to, those described in [Matioli, Elison & Brinkley, Stuart & Kelchner, Kathryn & Hu, Yan-Ling & Nakamura, Shuji & Denbaars, Steven & Speck, James & Weisbuch, C. (2012). High-brightness polarized light-emitting diodes. Light: Science & Applications. 1. 10.1038/lsa.2012.22]. In other embodiments, the first and second polarization states may be right-handed and left-handed polarization, respectively. Examples of LEDs which emit circularly polarized light include, but are not limited to, GaAs-based spin-polarized light-emitting diodes of the type described in [Nishizawa, Nozomi & Nishibayashi, Kazuhiro & Munekata, Hiro. (2016). Pure circular polarization electroluminescence at room temperature with spin-polarized light-emitting diodes. Proceedings of the National Academy of Sciences. 114. 0.1073/pnas.1609839114]. The CP LEDs of Nishizawa et al. may be fashioned as either right- or left-handed CPs through selection of the direction of magnetization of the spin injector.

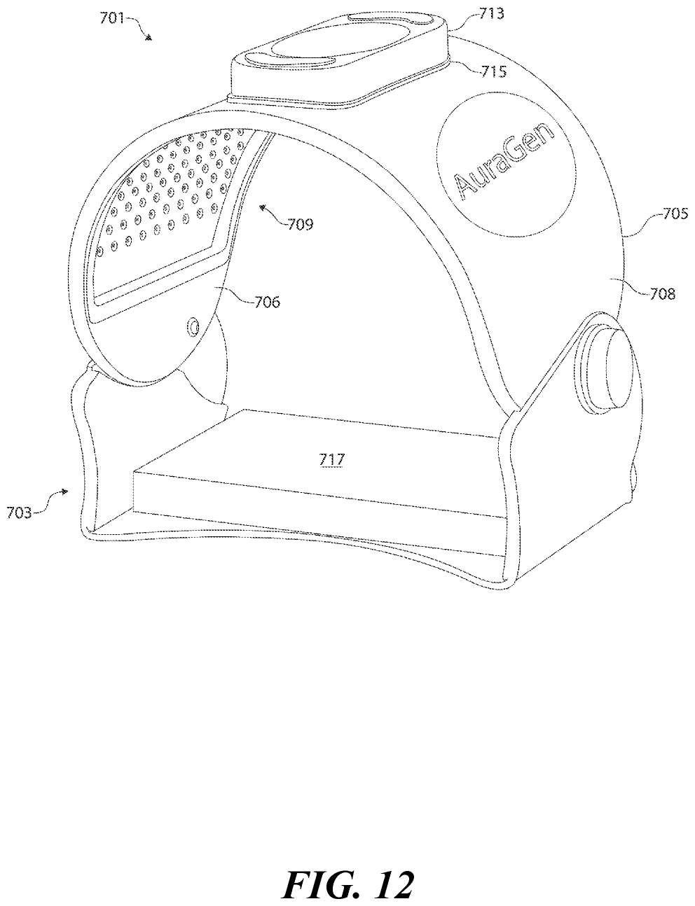

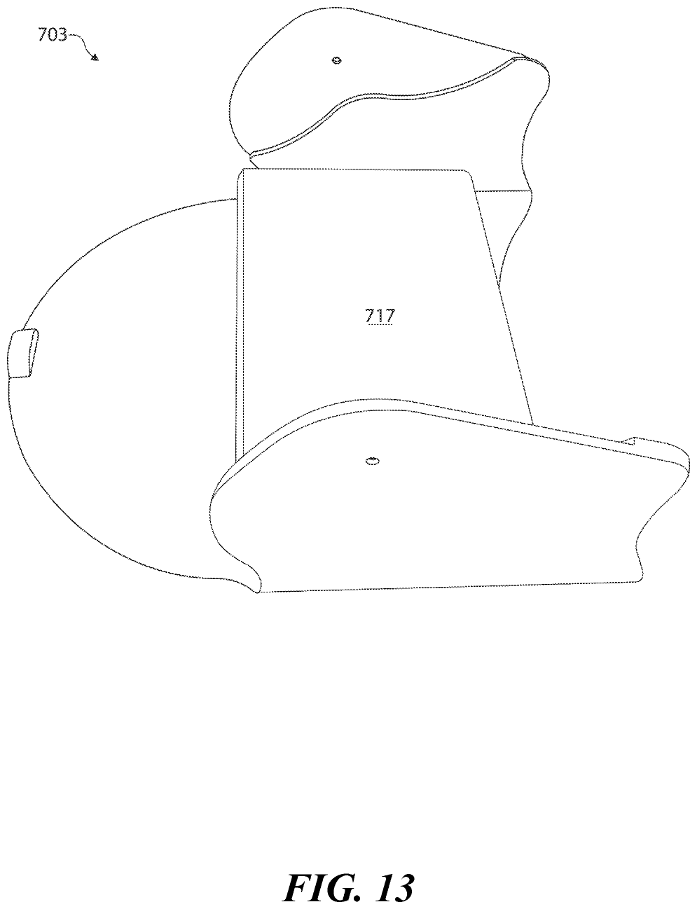

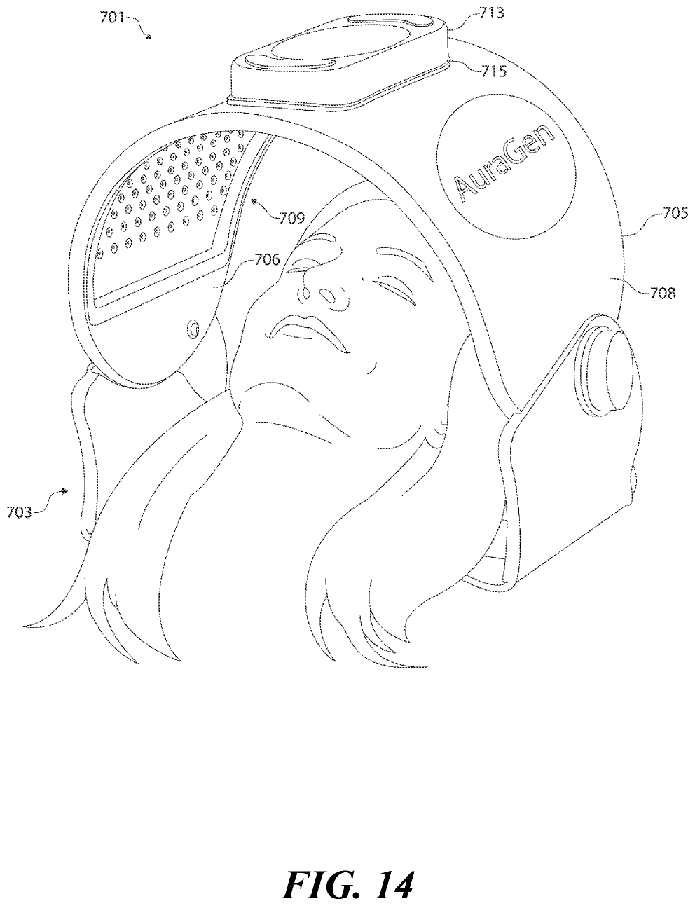

FIGS. 12-15 depict a first particular, non-limiting embodiment of a light therapy device in accordance with the teachings herein. With reference to FIG. 12, the light therapy device 101 comprises a base 103 (shown in isolation in FIG. 13) having a peripheral element 105 attached thereto and, optionally, an audio headset (not shown; the need for a headset may be determined, for example, by whether the entrainment methodology uses traveling waves originating from the same source, or standing waves generated by two distinct sources). The base 103 and peripheral element 105 define an opening 107 in which a user's head is placed (see FIG. 14). The base 103 and/or peripheral element 105 may be equipped with an audio jack, a Bluetooth transmitter, or other suitable provisions as necessary or desirable to support the use of an audio headset by the user. The base 103 is also equipped with a pillow 117 to support the head of the user.

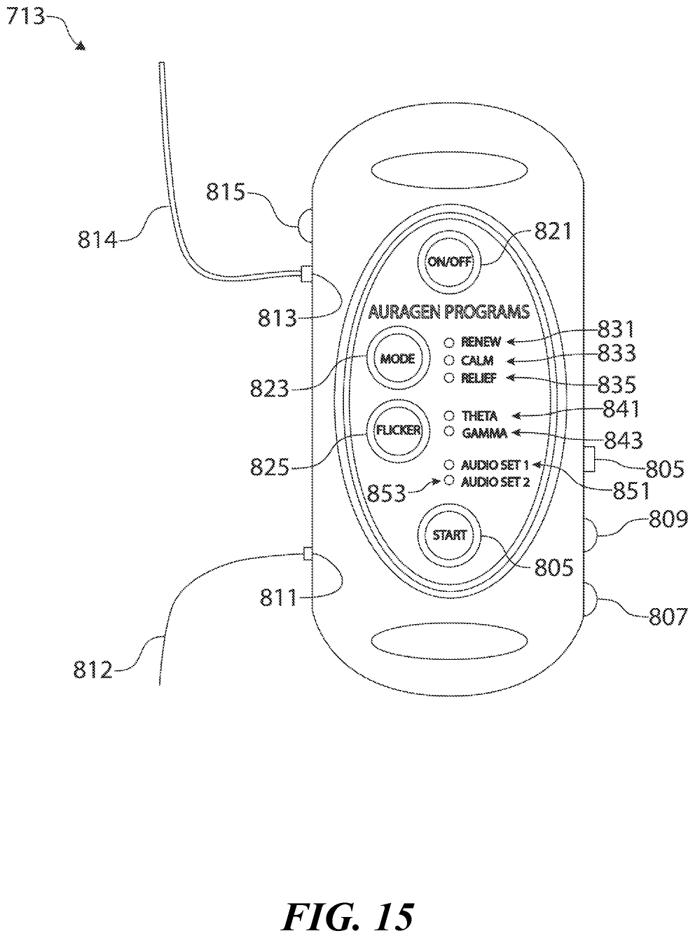

The base 103 in this particular embodiment is equipped with a pillow 111 for user comfort, and to provide the user with the ability to lie down or sleep during a brainwave entrainment session. The peripheral element 105 has a first major inward-facing surface 106 and a second major outward-facing surface 108. The first major surface 106 is equipped with an LED array 109 which can be activated with a remote control 113 to illuminate the user's head at one or more wavelengths. The second major surface 108 is equipped with a holder 115 for the remote control 113. The remote control 113, which is shown in greater detail in FIG. 15, may also be utilized to modulate the light emitted by the LED array 109, to select one or more wavelengths of light emitted by the LED array 109, and to control the playback of one or more audio files or tracks.

In normal use, a user's head is placed in the opening 107 such that the back of the user's head is on the pillow 111 and such that the user is facing the first major surface 106 of the peripheral portion 105 as shown in FIG. 14. The user (or possibly a clinician or other assistant) then uses the remote control 113 to activate the light therapy device 101 and to cause it to function in one or more selected modes. Regarding the latter, it is to be noted that the light therapy device 101 may be programmed with various algorithms which cause it to function in particular ways, some of which are described in greater detail below. The light therapy device 101 may also be programmed to play music or soundtracks, which may be advantageously matched to the particular algorithm being implemented by the light therapy device 101.

In some embodiments, the entrainment device may include a port to allow plugin of additional LED portable devices that operate in concert with the light therapy device 101 to provide light therapy to specific parts of the body. For example, such a portable LED device may be adapted to be positioned in the mouth of the user (via, for example, a mouth guard). In other embodiments, the entrainment device may include a small pad that may be wrapped or directly applied to a specific body part of the user. In still other embodiments, the entrainment device may include a set of googles or glasses that are placed over the eyes of the user to provide focused treatment to those areas, or to prevent treatment of those areas. Of course, it will be appreciated that any of the foregoing accessories may be utilized in combination in various embodiments of the systems and methodologies disclosed herein.

Various LEDs 109 or other light sources which emit at various wavelengths may be utilized in the devices and methodologies disclosed herein. However, the use of light sources which emit at wavelengths in the red, infra-red and blue-turquoise regions of the spectrum are preferred, and the use of light sources which emit at about 470 nm, 670 nm and 870 nm are especially preferred. In a preferred mode of operation, these light sources are made to oscillate or flicker in the theta or gamma band.

It will be appreciated that light may be emitted at the foregoing wavelengths in various manners, including sequentially or simultaneously. For example, the LED array 109 may be operated to emit electromagnetic radiation at a single wavelength (i.e., monochromatically) or at multiple wavelengths. In some cases, the LED array 109 may include a first set of LEDs that are operated to emit light at a first wavelength, a second set of LEDs that are operated to emit light at a second wavelength, and (optionally) a third set of LEDs that are operated to emit light at a third wavelength. In other cases, the LED array 109 may be operated such that all of the LEDs in the array emit light at a first wavelength for a first period of time, all of the LEDs in the array emit light at a second wavelength for a second period of time, and (optionally) all of the LEDs in the array emit light at a third wavelength for a third period of time.

The particular wavelength(s) of emission of the LED array 109, the duration of those emissions, the frequency of oscillation (if any), the intensity of the emitted light, the selection of accompanying audio tracks or files (if any), and/or the oscillation of any accompanying audio tracks, files or component(s) thereof, may be selected to achieve a desired physiological or psychological effect. It will be appreciated that, in some embodiments, the duration of emission for any particular wavelength of light may remain constant or may vary during the course of a therapy session. It will further be appreciated that, in some embodiments, any of the LEDs in the LED array 109 may be operated to emit two or more wavelengths of light, including broadband radiation or white light.

FIG. 15 depicts a particular, non-limiting embodiment of a remote control 713 that may be utilized with the light therapy device 701 of FIGS. 12-14. The remote control 713 comprises a body 801 which houses the electronics of the remote control 813, which will typically include an appropriate chipset and other suitable control circuitry. The remote control 713 is equipped with a central keypad 803 and peripheral controls, the latter of which include a track selection 805 for selecting one of a plurality of prerecorded audio tracks, a first volume control 807 for adjusting the audio volume of the selected audio track, and a second volume control 809 for controlling the volume of a second soundtrack featuring a sound at a specific frequency (for example, a gamma or beta frequency), which may be a diurnal beat. The two soundtracks may be played together or independently of each other.

The remote control 713 is further equipped with a headset audio plug-in port 811 for connecting a wired headset 812 to the remote control 713, and a power plug-in port 813 for connecting a power cord 814 to the remote control 713. The power cord 814 may be utilized to power the remote control 713 or to recharge one or more internal batteries contained within the device. The remote control 713 is also equipped with an LED indicator 815 to indicate when it is in a powered-on state.

The central keypad 803 includes an on/off button 821 which turns the remote control 113 on and off. A mode button 823 allows the user to toggle among mode selections (here, “Renew” 831, “Calm” 833 and “Relief” 835 mode selections), wherein each mode operates the light therapy device 101 in accordance with a particular program. A flicker button 825 allows the user to toggle among flicker settings. In the particular embodiment depicted, the flicker button 825 allows the user to select flickering at theta 841 or gamma 843 frequencies, or to deactivate flickering altogether. In the particular embodiment depicted, the central keypad 803 also includes audio set indicators which track which of a plurality of audio sets (here, audio set 1 851 and audio set 2 853) the track selection button 805 is sampling audio tracks from.

The brainwave entrainment devices and methodologies disclosed herein may be utilized as an effective tool in treating a subject for certain psychological or physiological conditions, or for prevention of these conditions. These conditions include, but are not limited to, traumatic brain injury, addiction or dependence (including, for example, addiction to, or dependence on, opioids, amphetamines, stimulants, alcohol or cannabis), depression (and more specifically, clinical depression or major depression), PTSD, developmental trauma disorder, traumatic brain injury and its sequelae, and Alzheimer's disease. In a preferred embodiment of the methodology disclosed herein, a subject is first diagnosed as suffering from one of the foregoing conditions, and then brainwave entrainment is utilized to treat the subject.

Various aspects of the systems and methodologies described herein have been described above with respect to the particular, non-limiting embodiments disclosed herein. It will be appreciated that these various aspects may be employed in various combinations (including various sub-combinations) or permutations in accordance with the teachings herein.

For example, while the use of light sources which emit at wavelengths in the red, infra-red and blue-turquoise regions of the spectrum are preferred, and the use of light sources which emit at about 470 nm, 670 nm and 870 nm are especially preferred, it will be appreciated that the devices and methodologies disclosed herein may utilize various other frequencies or wavelengths of electromagnetic radiation to achieve desired physiological or psychological effects. These wavelengths or frequencies may be selected, for example, from the visible, infrared or ultraviolet regions of the electromagnetic spectrum.

Similarly, in a preferred mode of operation, the intensities of one or more of these light sources are made to oscillate or flicker in the theta or gamma frequency band during at least a portion of a therapy session. However, embodiments are possible in which the light sources are made to oscillate or flicker at other frequencies, or in which the light sources (or elements thereof) operate in a manner which is not time varying. Embodiments are also possible in which the light sources are made to oscillate or flicker at harmonics of the foregoing frequencies.

While the embodiment of FIGS. 12-15 is a preferred embodiment of the brainwave entrainment device described herein, it will be appreciated that brainwave entrainment devices of various shapes, configurations, layouts and functionalities may be utilized in the practice of the methodologies disclosed herein, and these light therapy units may be provided with various accessories.

For example, in some embodiments, brainwave entrainment devices may be utilized that are adapted to illuminate one or more inner surfaces of a subject's oral cavity. In such embodiments, a light therapy unit utilized for this purpose may be fashioned as a standalone device, while in other embodiments, such a light therapy unit may be fashioned as an accessory to a main light therapy unit which is utilized to illuminate the outer surfaces of a subject's head. In embodiments of the latter type, the accessory may be adapted to communicate with the main brainwave entrainment device such that the accessory is controlled by, or acts in concert with, the main brainwave entrainment device.

In some instances of embodiments of a brainwave entrainment device adapted to illuminate one or more inner surfaces of a subject's oral cavity, the light therapy unit may be equipped with a mouth guard which is in optical communication with a light source by way of a suitable light guide, and which distributes light received from the light source in a suitable manner. In some cases, the mouthguard may be customized to the user. By way of example but not limitation, such a mouth guard may be adapted to direct suitable wavelengths of light to various surfaces of the oral cavity of a subject, including the teeth, gums, upper or lower mouth, and throat. The mouth guard, light guide or portions thereof may be equipped with suitable materials that specularly or diffusely transmit or reflect incident radiation in one or more directions. In addition to their possible use in treating physiological or psychological conditions, these embodiments may offer additional benefits such as, for example, the treatment or prevention of gingivitis and other bacterial infections.

In some embodiments of the devices disclosed herein, measures may be taken to ensure that the brainwave entrainment device is applied to only specific parts of the user's body. For example, in some embodiments, the aforementioned light therapy unit which is adapted to illuminate one or more inner surfaces of a subject's oral cavity may be used by itself such that only these surfaces are exposed to the brainwave entrainment therapy. Similarly, in some embodiments, the user may be equipped with glasses or goggles such that the user's eyes or optical nerves are not exposed to the brainwave entrainment light, or such that this light is concentrated on the user's eyes or optical nerves. In still other embodiments, an optical pad or other suitable means may be utilized to apply brainwave entrainment device only to the back of a user's neck, or to a user's chest (alone or in combination with the application of entraining frequencies to the user's head).

Preferred embodiments of the devices disclosed herein are adapted to allow the user to lie down or otherwise assume a state of repose during a brainwave entrainment session. Such embodiments may include, for example, a pillow or one or more deformable pads which support the user's head during brainwave entrainment therapy. Here, it is notable that many other devices in the art which are designed for brainwave entrainment therapy require the user to remain in a sitting or standing position for the duration of the therapy.

In some embodiments of the devices disclosed herein, the device may be equipped with a suitable controller, which may be wireless or wired. The controller may be programmable or pre-programmed, and may be equipped with suitable programming instructions (which may include an operating system) recorded in a tangible, non-transient medium that cause the brainwave entrainment device to operate in various modes or to perform various functions. These modes or functions may be selected or optimized for the treatment of various portions of a subject's body, or for the treatment of particular physiological or psychological conditions.

Various parameters (and ranges of these parameters) may be utilized in the brainwave entrainment devices and methodologies disclosed herein. These include, without limitation, wavelength, frequency, entrainment waveform, energy, fluence, power, irradiance, intensity, pulse mode, treatment duration, and repetition. These parameters and their values may be selected to treat a subject for certain psychological or physiological conditions, to lessening the severity or effects of these conditions, and/or to preventing the occurrence of these conditions. These conditions include, but are not limited to, traumatic brain injury, opioid addiction (including, for example, heroin addiction or addiction to prescription opioids), alcohol misuse disorder or alcohol dependence, nicotine dependence or addiction, depression (and more specifically, clinical depression or major depression), mild cognitive impairment, dementia, Alzheimer's disease, attention deficit disorder, developmental trauma disorder, and autism.

It will be appreciated that the brainwave entrainment devices disclosed herein, and the components thereof, may be equipped with suitable optical elements to achieve various purposes. Such optical elements (or portions thereof) may be diffusely or specularly reflective or transmissive. Suitable optical elements may include, but are not limited to, reflective elements, polarizers, color shifting elements, filters, light guides (including, without limitation, optical fibers, light pipes and waveguides), prismatic elements, lenses (including Fresnel lenses), and lens arrays.

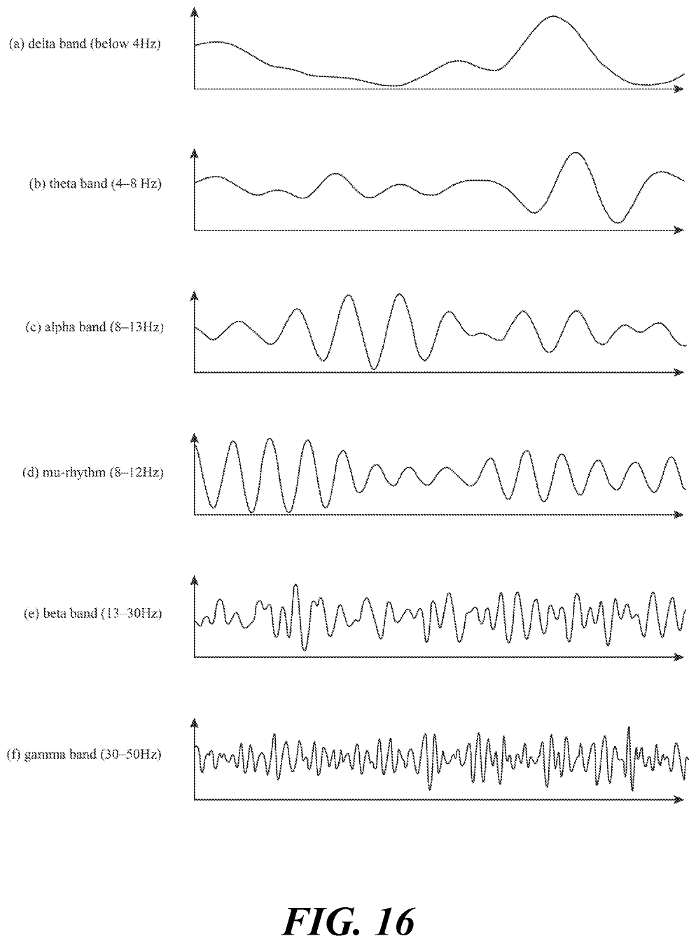

In preferred embodiments of the systems and methodologies disclosed herein, one or more audio tracks or audio files may be provided that may be modulated, coordinated and/or synchronized with the plurality of LEDs or the light emitted therefrom. Preferably, the audio tracks or audio files include sound that is modulated, coordinated and/or synchronized with the LEDs or the light emitted therefrom at one or more frequencies selected from the ranges depicted in FIG. 16. The audio tracks or files (alone, or in combination with any light wavelengths utilized) may be selected to achieve a desired physiological or psychological effect in the user, either alone or in combination with the light therapy.

One skilled in the art will further appreciate that the systems and methodologies disclosed herein may be used not only to treat various physiological or psychological conditions, but to prevent them from occurring in the first place. For example, these systems and methodologies may be adapted to prophylactically prevent the onset of depression, PTSD, ADHD, opioid addiction (for example, heroine or oxycodone), or conditions resulting from traumatic brain injury, or of conditions which might otherwise result from the foregoing.

The systems and methodologies disclosed herein may be utilized in conjunction with other methodologies or techniques. For example, these systems and methodologies may be used in combination with emotional freedom technique (EFT) tapping. EFT tapping is a holistic healing technique that may be utilized to treat various issues including, without limitation, stress, anxiety, phobias, emotional disorders, chronic pain, addiction, weight control, and limiting beliefs. EFT tapping involves tapping with the fingertips on specific meridian endpoints of the body, while focusing on negative emotions or physical sensations. Proponents of the method claim that it calms the nervous system, rewires the brain to respond in healthier ways, and restores the body's balance of energy.

One skilled in the art will further appreciate that the optimal parameters for a brainwave entrainment session may depend on a variety of factors including, but not limited to, the condition being treated (or prevented), the physiological or psychological state of the user, the user's biometrics, and other such factors. In some use cases, selection of these parameters may be made by, or in coordination with, a physician, a psychiatrist, or other healthcare provider. These parameters may include, but are not limited to, the wavelengths of light to be utilized, the audio tracks or files to accompany the light therapy, the frequencies of oscillation utilized for the intensity in any of the wavelengths or light or sound, the portions of the user's head or body to be exposed to the light therapy, and the duration of the treatment.

While the devices and methodologies disclosed herein have frequently been described with reference to the use of traveling waves originating from a common source, one skilled in the art will appreciate that various embodiments of these methodologies and devices may also be produced which utilize waves originating from distinct sources (e.g., standing waves). In some embodiments, various devices, materials or other such measures may be taken to cause or prevent reflection of the waves used for brainwave entrainment.

The devices and methodologies disclosed herein have frequently been described or illustrated with respect to light therapy devices. However, it is to be understood that these devices and methodologies may have many uses in other fields and applications. These include, without limitation, their use in photic stimulation, including intermittent photic stimulation.

It will be appreciated that the polarizers and polarizing techniques utilized in the devices and methodologies disclosed herein may produce electromagnetic radiation that is less than 100% polarized. Typically, where polarized electromagnetic radiation is called for, the electromagnetic radiation is at least 60% polarized, preferably at least 70% polarized, more preferably at least 80% polarized, even more preferably at least 90% polarized, and most preferably at least 95% polarized.

In some embodiments of the methodologies disclosed herein, the various techniques disclosed herein for performing light therapy with light having an oscillating polarization state may be applied to brainwave entrainment. In some embodiments, the subject of the brainwave entrainment (or, in some cases, an individual distinct from the subject) may be made to perform a mental task. Such a task may include, for example, the simultaneous maintenance of multiple items in working memory as may be implemented, for example, in a complex maze test. See, e.g., [Argento, E., Papagiannakis, G., Baka, E., Maniadakis, M., Trahanias, P., Sfakianakis, M., Nestoros, I., 2017. Augmented Cognition via Brainwave Entrainment in Virtual Reality: An Open, Integrated Brain Augmentation in a Neuroscience System Approach. Augmented Human Research 2. doi:10.1007/s41133-017-0005-3], which is incorporated herein by reference in its entirety. An EEG of the subject or individual may be taken during performance of the task, and one or more (preferably dominant) brainwaves may be identified from the EEG. The one or more identified brainwaves may then be utilized to perform brainwave entrainment on the subject. In some embodiments, the one or more identified brainwaves may include first and second brainwaves, and brainwave entrainment may be performed on the subject using a nested waveform of which the first and second brainwaves are components.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. It will also be appreciated that the various features set forth in the claims may be presented in various combinations and sub-combinations in future claims without departing from the scope of the invention. In particular, the present disclosure expressly contemplates any such combination or sub-combination that is not known to the prior art, as if such combinations or sub-combinations were expressly written out.

1-83. (canceled)

84. A method for performing electromagnetic radiation therapy on a subject, comprising:
(a) providing an electromagnetic radiation fixture equipped with an LED array containing (i) a first set of LEDs which emit electromagnetic radiation in a first polarization state, and (ii) a second set of LEDs which emit electromagnetic radiation in a second polarization state;
(b) positioning the electromagnetic radiation fixture such that electromagnetic radiation emitted by the fixture is directed at the subject; and
(c) oscillating the LED array between first and second illumination states selected from the group consisting of
(j) a first illumination state in which the first set of LEDs are illuminated and the second set of LEDs are not illuminated, and a second illumination state in which the first set of LEDs are not illuminated and the second set of LEDs are illuminated,
(jj) a first illumination state in which the first set of LEDs are powered on and the second set of LEDs are powered off, and a second illumination state in which the first set of LEDs are powered off and the second set of LEDs are powered on,
(jjj) a first illumination state in which the power supply to the first set of LEDs is at a maximum and the power supplied to the second set of LEDs is at a minimum, and a second illumination state in which the power supply to the first set of LEDs is at a minimum and the power supply to the second set of LEDs is at a maximum, and
(jjjj) a first illumination state in which the current supplied to the first set of LEDs is hi and the power supplied to the second set of LEDs is I₁₂, and a second illumination state in which the power supply to the first set of LEDs is I₂₁ and the power supply to the second set of LEDs is I₂₂, wherein I₁₁>I₂₁ and I₁₂<I₂₂.

85. The method as set forth in claim 84, wherein the electromagnetic radiation emitted by the electromagnetic radiation fixture is unpolarized in the first polarization state, and is polarized in the second polarization state.

86. The method as set forth in claim 84, wherein the electromagnetic radiation is linearly polarized in the first polarization state and has an electric field which is confined to a first plane that is orthogonal to the direction of propagation of the electromagnetic radiation in the first polarization state, wherein the electromagnetic radiation is linearly polarized in the second polarization state and has an electric field which is confined to a second plane that is orthogonal to the direction of propagation of the electromagnetic radiation in the second polarization state, and wherein the first and second planes are not coplanar.

87. The method as set forth in claim 84, wherein the electromagnetic radiation is circularly polarized in the first polarization state and has an electric field which is confined to a first plane that is orthogonal to the direction of propagation of the electromagnetic radiation in the first polarization state, wherein the electromagnetic radiation is circularly polarized in the second polarization state and has an electric field which is confined to a second plane that is orthogonal to the direction of propagation of the electromagnetic radiation in the second polarization state, and wherein the first and second planes are not coplanar.

88. The method as set forth in claim 84, wherein the electromagnetic radiation is elliptically polarized in the first polarization state and has an electric field which is confined to a first plane that is orthogonal to the direction of propagation of the electromagnetic radiation in the first polarization state, wherein the electromagnetic radiation is elliptically polarized in the second polarization state and has an electric field which is confined to a second plane that is orthogonal to the direction of propagation of the electromagnetic radiation in the second polarization state, and wherein the first and second planes are not coplanar.

89. The method as set forth in claim 84, wherein the electromagnetic radiation is linearly polarized in the first polarization state, and is circularly polarized in the second polarization state.

90. The method as set forth in claim 84, wherein the electromagnetic radiation is circularly polarized in the first polarization state, and is elliptically polarized in the second polarization state.

91. The method as set forth in claim 84, wherein the electromagnetic radiation is circularly polarized in a left-handed orientation in the first polarization state, and is circularly polarized in a right-handed orientation in the second polarization state.

92. The method as set forth in claim 84, wherein the electromagnetic radiation is elliptically polarized in a left-handed orientation in the first polarization state, and is elliptically polarized in a right-handed orientation in the second polarization state.

93. The method as set forth in claim 84, wherein the electromagnetic radiation is elliptically polarized in a left-handed orientation in the first polarization state, and is circularly polarized in a left-handed orientation in the second polarization state.

94. The method as set forth in claim 84, wherein the electromagnetic radiation is elliptically polarized in a left-handed orientation in the first polarization state, and is circularly polarized in a right-handed orientation in the second polarization state.

95. The method as set forth in claim 84, wherein the electromagnetic radiation fixture is further equipped with a polarizer array disposed over the LED array, wherein the polarizer array includes a plurality of polarizers, and wherein each LED in the LED array has one of said plurality of polarizers disposed in an output optical path of the LED.

96. The method as set forth in claim 84, wherein the first illumination state is a state in which the first set of LEDs are illuminated and the second set of LEDs are not illuminated, and wherein the second illumination state is a state in which the first set of LEDs are not illuminated and the second set of LEDs are illuminated.

97. The method as set forth in claim 84, wherein the first illumination state is a state in which the first set of LEDs are powered on and the second set of LEDs are powered off, and wherein the second illumination state is a state in which the first set of LEDs are powered off and the second set of LEDs are powered on.

98. The method as set forth in claim 84, wherein the first illumination state is a state in which the power supply to the first set of LEDs is at a maximum and the power supplied to the second set of LEDs is at a minimum, and wherein the second illumination state is a state in which the power supply to the first set of LEDs is at a minimum and the power supply to the second set of LEDs is at a maximum.

99. The method as set forth in claim 84, wherein the first illumination state is a state in which the current supplied to the first set of LEDs is hi and the power supplied to the second set of LEDs is I₁₂, and wherein the second illumination state is a state in which the power supply to the first set of LEDs is I₂₁ and the power supply to the second set of LEDs is I₂₂, wherein I₁₁>I₂₁ and I₁₂<I₂₂.

100. The method as set forth in claim 84, further comprising:
(a) determining at least one brainwave frequency which is associated with a mental task; and
(b) performing brainwave entrainment on the subject using the at least one brainwave frequency by oscillating the LED array between the first and second illumination states at the at least one brainwave frequency.

101. The method as set forth in claim 100, wherein the at least one frequency includes first and second frequencies, wherein the first and second frequencies are components of a nested waveform, and wherein brainwave entrainment on the subject is performed using the nested waveform.

102. The method as set forth in claim 100, wherein determining at least one brainwave frequency which is associated with a mental task includes:
(a) obtaining an electroencephalogram (EEG) from the subject while the subject is performing the mental task; and
(b) determining the at least one brainwave frequency from the EEG.

103. The method as set forth in claim 100, wherein the mental task includes the simultaneous maintenance of multiple items in working memory.
